(19) United States
(12) Patent Application Publication
(10) Pub. No.: US 2024/0131188 A1
(43) Pub. Date: Apr. 25, 2024

(54) MODULATION OF GENE EXPRESSION FOR DISEASE TREATMENT

(71) Applicant: Dogodan Therapeutics, Inc., Westford, MA (US)

(72) Inventors: Thomas T. Tibbitts, Westford, MA (US); Diego Borges-Rivera, Belmont, MA (US)

(21) Appl. No.: 18/497,176

(22) Filed: Oct. 29, 2023

Related U.S. Application Data
(63) Continuation of application No. PCT/US2022/027128, filed on Apr. 29, 2022.
(60) Provisional application No. 63/182,631, filed on Apr. 30, 2021.

(51) Int. Cl. A61K 48/00; C07K 14/47; C12N 9/22; C12N 15/115

(52) U.S. Cl. CPC A61K 48/005; C07K 14/4702; C12N 9/22; C12N 15/115; C12N 2310/20

(57) ABSTRACT

Provided in the present disclosure include targeted therapeutics for regulating gene transcription at a specific locus (or loci). Such targeted therapeutics can be used as novel genomic therapeutics for treating and/or preventing a disease or disorder that is tightly associated with the locus where the gene transcription is reprogrammed using the present systems. The regulatory system comprises a macromolecular complex of transcription effector proteins that is directed to a locus using CRISPR-dCas9.

### RELATED APPLICATIONS

The instant application is a Continuation of International Application No. PCT/US2022/027128, filed Apr. 29, 2022, and which claims priority to U.S. Provisional Application No. 63/182,631, filed Apr. 30, 2021. The entire contents of each of the foregoing applications are expressly incorporated by reference herein in their entireties.

### SEQUENCE LISTING

The application contains a Sequence Listing which has been submitted electronically in .XML format and is hereby incorporated by reference in its entirety. Said .XML copy, created on Oct. 27, 2023, is named “135700-00102.xml” and is 34,422 bytes in size. The sequence listing contained in this .XML file is part of the specification and is hereby incorporated by reference herein in its entirety.

### FIELD OF THE DISCLOSURE

The present disclosure relates to novel targeted therapeutics. Provided also include a novel platform for designing targeted therapeutics for gene regulation. In particular, the present disclosure relates to novel therapeutics for respiratory diseases and viral infections such as, but not limited to, COVID-19.

### BACKGROUND OF THE DISCLOSURE

Developing a method to modify disease processes at the level of gene expression has the potential to treat diseases that cannot currently be drugged by small molecules or biologics (e.g., antibodies). The advent of the CRISPR (clustered regularly interspaced short palindromic repeats)/Cas9 (CRISPR-associated protein 9) system ushered in a new era in cell biology by providing a relatively simple technology for editing the genetic blueprint in a cell at a specific site. The CRISPR/Cas9 system allows researchers to not only specifically correct disease-causing errors in the DNA code (e.g., gene mutations), it also has been adapted for modulating the biology of the cell by changing expression of a gene or set of genes. These two therapeutic strategies have been employed across a wide range of indications (e.g., Yoshida, et al., Development of an integrated CRISPRi targeting ΔNp63 for treatment of squamous cell carcinoma, Oncotarget 2018, 9:29220-29232); Kemaladewi, et al., A mutation-independent approach for muscular dystrophy via upregulation of a modifier gene, Nature 2019, 572:125-130); Matharu, et al. CRISPR-mediated activation of a promoter or enhancer rescues obesity caused by haploinsufficiency, Science, 2019, 363: 231; and Moses, et al. Activating PTEN tumor suppressor expression with the CRISPR/dCas9 system, Mol. Ther. Nucleic Acids., 2019, 14:287-300).

The present disclosure provides a next generation of genomics-based therapeutics for such need. The present next generation of genomics-based therapeutics is composed of a modular set of self-assembling macromolecular machines that are recruited to specific regions of a target locus in a genome and selectively control the expression of a gene, or a set of genes at that locus. The therapeutics integrate CRISPR/dCas9 technologies with technologies involving interaction of RNAs and small RNA binding polypeptides, to position the machinery required to achieve a desired gene transcription to a genetic region associated with a disease or disorder.

The components of the novel self-assembling macromolecular complex are designed to have exquisite selectivity for a single genetic locus to minimize off-target effects, providing a way of drugging targets which are not accessible to traditional therapeutic modalities such as small molecules and antibodies. The platform disclosed has the potential to have a revolutionary impact on medicine in that it is broadly applicable for a wide range of human diseases while remaining highly personalized.

### SUMMARY OF THE DISCLOSURE

The present disclosure provides a novel platform for designing genomics-based targeted therapeutics to provide highly personalized medicines for a wide range of human diseases.

In one aspect this disclosure provides a method of designing a targeted therapeutic, comprising: a) selecting one or more gene sequences encoding one or more effector proteins or fragments thereof; and b) selecting a gene sequence encoding one or more RNA-guided molecules and a gene sequence encoding one or more guide RNAs (gRNA), wherein the RNA-guided molecule and gRNA interact with a target nucleic acid in the genome, and wherein the one or more effector proteins or fragments thereof are guided by the RNA-guided molecule/gRNA complex to the target nucleic acid in the genome.

In some embodiments, the targeted therapeutic comprises one or more nucleic acid constructs. In some embodiments, the RNA-guided molecule comprises a dead Cas9 (dCas9) protein.

In some embodiments, the dCas9 is selected from the group consisting of SpCas9, SaCas9, NmeCas9, BlCas9, CdCas9, ClCas9, CjCas9, FnCas9, LiCas9, nCas9RHA, NcCas9, PlCas9, PmCas9, SmCas9, SpaCas9, St1Cas9, St3Cas9, TdCas9, LbCpf1 and AsCpf1.

In some embodiments, the gRNA comprises either 1) a crispr RNA (crRNA) and a trans-activating crispr RNA (tracrRNA), or 2) a single guide RNA (sgRNA). In some embodiments, the gRNA is a sgRNA. In some embodiments, the sgRNA comprises a targeting domain complimentary to the target nucleic acid in the genome and a Cas9-binding domain, wherein dCas9 is capable of binding the Cas9-binding domain.

In some embodiments, the gRNA further comprises one or more RNA aptamers. In some embodiments, the one or more RNA aptamers are capable of binding one or more RNA-binding polypeptides.

In some embodiments, the RNA-guided molecule further comprises one or more RNA-binding polypeptides. In some embodiments, the RNA-guided molecule is covalently linked to the one or more RNA-binding polypeptides.

In some embodiments, the target nucleic acid in the genome is non-coding DNA at or adjacent to one or more target genes. In some embodiments, the non-coding DNA includes cis-regulatory elements (CREs).

In some embodiments, expression of the one or more target genes are regulated by the one or more effector proteins or fragments thereof. In some embodiments, the expression of the one or more target genes is activated or repressed.

In some embodiments, the one or more effector proteins or fragments thereof are covalently linked with one or more RNA-binding polypeptides.

In some embodiments, the method further comprising: 1) selecting one or more gene sequences encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or 2) selecting one or more gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or 3) selecting one or more gene sequences encoding a third RNA molecule, wherein the third RNA molecule comprises one or more RNA aptamers and wherein the third RNA molecule binds to the t or more RNA-binding polypeptides covalently linked to two or more RNA-binding polypeptides.

In some embodiments, in a cell the targeted therapeutic expresses 1) one or more effector proteins or fragments thereof covalently linked to one or more RNA-binding polypeptides; 2) one or more RNA-guided molecule covalently linked to one or more RNA-binding polypeptides and one or more guide RNA (gRNA); and 3) one or more RNA molecules, wherein the one or more RNA molecules binds to a) one or more RNA-guided molecule and one or more effector proteins or fragments thereof; b) one or more effector proteins or fragments thereof; and/or c) one or more RNA-guided molecules.

In some embodiments, in a cell: 1) the one or more RNA-guided molecule is guided to the target nucleic acid in the genome by the one or more guide RNA (gRNA); 2) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; 3) the one or more effector proteins or fragments thereof regulate the target nucleic acid.

In some embodiments, the one or more effector proteins or fragments thereof are selected from the group consisting of: transcription factors, DNA modification enzymes and cofactors, and histone modifying enzymes and cofactors, and epigenetic regulators.

In some embodiments, one or more effector proteins or fragments thereof are an entire protein, a fusion protein, an effector domain of the effector protein, or covalently linked effector domains from one or more effector proteins or fragments thereof, or a combination thereof.

In some embodiments, the components of the targeted therapeutic are designed based on 3D structural modeling. In some embodiments, the targeted therapeutic is inducible.

In some embodiments, the targeted therapeutic is present in a composition. In some embodiments, the composition comprises a pharmaceutical composition. In some embodiments, the pharmaceutical composition comprises a lipid formulation.

In some embodiments, the lipid formulation comprises one or more cationic lipids, non-cationic lipids, and/or PEG-modified lipids, or a combination thereof. In some embodiments, the pharmaceutical composition comprises a lipid nanoparticle.

In another aspect, the invention provides a targeted therapeutic comprising: a) one or more gene sequences encoding one or more effector proteins, or fragments thereof; and b) a gene sequence encoding one or more RNA-guided molecules and a gene sequence encoding one or more guide RNAs (gRNA), wherein the RNA-guided molecule and gRNA interact with a target nucleic acid in the genome, and wherein the one or more effector proteins or fragments thereof are guided by the RNA-guided molecule/gRNA complex to the target nucleic acid in the genome.

In some embodiments, the targeted therapeutic comprises one or more nucleic acid constructs.

In some embodiments, the RNA-guided molecule comprises a dead Cas9 (dCas9) protein. In some embodiments, the dCas9 is selected from the group consisting of SpCas9, SaCas9, NmeCas9, BlCas9, CdCas9, ClCas9, CjCas9, FnCas9, LiCas9, nCas9RHA, NcCas9, PlCas9, PmCas9, SmCas9, SpaCas9, StlCas9, St3Cas9, TdCas9, LbCpf1 and AsCpf1.

In some embodiments, the gRNA comprises either 1) a crispr RNA (crRNA) and a trans-activating crispr RNA (tracrRNA), or 2) a single guide RNA (sgRNA). In some embodiments, the gRNA is a sgRNA. In some embodiments, the sgRNA comprises a targeting domain complimentary to the target nucleic acid in the genome and a Cas9-binding domain, wherein dCas9 is capable of binding the Cas9-binding domain.

In some embodiments, the gRNA further comprises one or more RNA aptamers. In some embodiments, the one or more RNA aptamers are capable of binding one or more RNA-binding polypeptides.

In some embodiments, the RNA-guided molecule further comprises one or more RNA-binding polypeptides. In some embodiments, the RNA-guided molecule is covalently linked to the one or more RNA-binding polypeptides.

In some embodiments, the target nucleic acid in the genome is non-coding DNA at or adjacent to one or more target genes. In some embodiments, the non-coding DNA includes cis-regulatory elements (CREs).

In some embodiments, expression of the one or more target genes are regulated by the one or more effector proteins or fragments thereof. In some embodiments, the expression of the one or more target genes is activated or repressed.

In some embodiments, the one or more effector proteins or fragments thereof are covalently linked with one or more RNA-binding polypeptides.

In some embodiments, the method further comprising: 1) one or more gene sequences encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or 2) one or more gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or 3) one or more gene sequences encoding a third RNA molecule, wherein the third RNA molecule comprises one or more RNA aptamers and wherein the third RNA molecule binds to the t or more RNA-binding polypeptides covalently linked to two or more RNA-binding polypeptides.

In some embodiments, in a cell the targeted therapeutic expresses 1) one or more effector proteins or fragments thereof covalently linked to one or more RNA-binding polypeptides; 2) one or more RNA-guided molecules covalently linked to one or more RNA-binding polypeptides and one or more guide RNAs (gRNA); and 3) one or more RNA molecules, wherein the one or more RNA molecules binds to a) one or more RNA-guided molecules and one or more effector proteins or fragments thereof; b) one or more effector proteins or fragments thereof; and/or c) one or more RNA-guided molecules.

In some embodiments, in a cell: 1) the one or more RNA-guided molecules is guided to the target nucleic acid in the genome by the one or more guide RNAs (gRNA); 2) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; 3) the one or more effector proteins or fragments thereof regulate the target nucleic acid.

In some embodiments, the one or more effector proteins or fragments thereof are selected from the group consisting of: transcription factors, DNA modification enzymes and cofactors, and histone modifying enzymes and cofactors, and epigenetic regulators.

In some embodiments, one or more effector or fragments thereof proteins are an entire protein, a fusion protein, an effector domain of the effector protein, or covalently linked effector domains from one or more effector proteins or fragments thereof, or a combination thereof.

In some embodiments, the components of the targeted therapeutic are designed based on 3D structural modeling. In some embodiments, the targeted therapeutic is inducible.

In some embodiments, the targeted therapeutic is present in a composition. In some embodiments, the composition comprises a pharmaceutical composition.

In some embodiments, the pharmaceutical composition comprises a lipid formulation. In some embodiments, the lipid formulation comprises one or more cationic lipids, non-cationic lipids, and/or PEG-modified lipids, or a combination thereof. In some embodiments, the pharmaceutical composition comprises a lipid nanoparticle.

In some embodiments, the pharmaceutical composition further comprising a lipid nanoparticle.

In another aspect, the invention provides a method of regulating gene transcription in a cell, comprising: contacting the cell with a targeted therapeutic, wherein the targeted therapeutic comprises i) a gene sequences encoding one or more effector proteins or fragments thereof; and ii) a gene sequence encoding one or more RNA-guided molecules and a gene sequence encoding one or more guide RNAs (gRNA); and wherein the RNA-guided molecule and gRNA interact with a target nucleic acid in the genome, and wherein the one or more effector proteins or fragments thereof are guided by the RNA-guided molecule/gRNA complex to the target nucleic acid in the genome.

In some embodiments, the targeted therapeutic comprises one or more nucleic acid constructs. In some embodiments, the RNA-guided molecule comprises a dead Cas9 (dCas9) protein. In some embodiments, the dCas9 is selected from the group consisting of SpCas9, SaCas9, NmeCas9, BlCas9, CdCas9, ClCas9, CjCas9, FnCas9, LiCas9, nCas9RHA, NcCas9, PlCas9, PmCas9, SmCas9, SpaCas9, StlCas9, St3Cas9, TdCas9, LbCpf1 and AsCpf1.

In some embodiments, the gRNA comprises either 1) a crispr RNA (crRNA) and a trans-activating crispr RNA (tracrRNA), or 2) a single guide RNA (sgRNA). In some embodiments, the gRNA is a sgRNA.

In some embodiments, the sgRNA comprises a targeting domain complimentary to the target nucleic acid in the genome and a Cas9-binding domain, wherein dCas9 is capable of binding the Cas9-binding domain.

In some embodiments, the gRNA further comprises one or more RNA aptamers. In some embodiments, the one or more RNA aptamers are capable of binding one or more RNA-binding polypeptides.

In some embodiments, the RNA-guided molecule further comprises one or more RNA-binding polypeptides. In some embodiments, the RNA-guided molecule is covalently linked to the one or more RNA-binding polypeptides.

In some embodiments, the target nucleic acid in the genome is non-coding DNA at or adjacent to one or more target genes. In some embodiments, the non-coding DNA includes cis-regulatory elements (CREs).

In some embodiments, expression of the one or more target genes are regulated by the one or more effector proteins or fragments thereof. In some embodiments, the expression of the one or more target genes is activated or repressed.

In some embodiments, the one or more effector proteins or fragments thereof are covalently linked with one or more RNA-binding polypeptides.

In some embodiments, the targeted therapeutic further comprises: 1) a gene sequence encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or 2) a gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or 3) a gene sequence encoding a third RNA molecule, wherein the third RNA molecule comprises one or more RNA aptamers and wherein the third RNA molecule binds to the one or more RNA-binding polypeptides covalently linked to two or more RNA-binding polypeptides.

In some embodiments, in the cell the targeted therapeutic expresses 1) one or more effector proteins or fragments thereof covalently linked to one or more RNA-binding polypeptides; 2) one or more RNA-guided molecules covalently linked to one or more RNA-binding polypeptides and one or more guide RNAs (gRNA); and 3) one or more RNA molecules, wherein the one or more RNA molecules binds to a) one or more RNA-guided molecules and one or more effector proteins or fragments thereof; b) one or more effector proteins or fragments thereof; and/or c) one or more RNA-guided molecules.

In some embodiments, in the cell: 1) the one or more RNA-guided molecules is guided to the target nucleic acid in the genome by the one or more guide RNAs (gRNA); 2) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; 3) the one or more effector proteins or fragments thereof regulate the target nucleic acid.

In some embodiments, the one or more effector proteins or fragments thereof are selected from the group consisting of: transcription factors, DNA modification enzymes and cofactors, and histone modifying enzymes and cofactors, and epigenetic regulators.

In some embodiments, one or more effector proteins or fragments thereof are an entire protein, a fusion protein, an effector domain of the effector protein, or covalently linked effector domains from one or more effector proteins or fragments thereof, or a combination thereof.

In some embodiments, the components of the targeted therapeutic are designed based on 3D structural modeling. In some embodiments, the targeted therapeutic is inducible.

In some embodiments, the cell is mammalian. In some embodiments, the cell is human.

In some embodiments, the targeted therapeutic is present in a composition. In some embodiments, the composition comprises a pharmaceutical composition. In some embodiments, the pharmaceutical composition comprises a lipid formulation.

In some embodiments, the lipid formulation comprises one or more cationic lipids, non-cationic lipids, and/or PEG-modified lipids, or a combination thereof.

In some embodiments, the pharmaceutical composition comprises a lipid nanoparticle.

In some embodiments, the targeted therapeutic/lipid particle is administered intranasally, intramuscularly, or intravenously.

In another aspect, the invention provides method of treating disease in a subject, a method comprising: administering to the subject a targeted therapeutic, wherein the targeted therapeutic comprises i) a gene sequences encoding one or more effector proteins or fragments thereof; and ii) a gene sequence encoding one or more RNA-guided molecules and a gene sequence encoding one or more guide RNAs (gRNA); and wherein the RNA-guided molecule and gRNA interact with a target nucleic acid in the genome, and wherein the one or more effector proteins or fragments thereof are guided by the RNA-guided molecule/gRNA complex to the target nucleic acid in the genome.

In some embodiments, the targeted therapeutic comprises one or more nucleic acid constructs. In some embodiments, the RNA-guided molecule comprises a dead Cas9 (dCas9) protein. In some embodiments, the dCas9 is selected from the group consisting of SpCas9, SaCas9, NmeCas9, BlCas9, CdCas9, ClCas9, CjCas9, FnCas9, LiCas9, nCas9RHA, NcCas9, PlCas9, PmCas9, SmCas9, SpaCas9, StlCas9, St3Cas9, TdCas9, LbCpf1 and AsCpf1.

In some embodiments, the gRNA comprises either 1) a crispr RNA (crRNA) and a trans-activating crispr RNA (tracrRNA), or 2) a single guide RNA (sgRNA). In some embodiments, the gRNA is a sgRNA.

In some embodiments, the sgRNA comprises a targeting domain complimentary to the target nucleic acid in the genome and a Cas9-binding domain, wherein dCas9 is capable of binding the Cas9-binding domain.

In some embodiments, the gRNA further comprises one or more RNA aptamers.

In some embodiments, the one or more RNA aptamers are capable of binding one or more RNA-binding polypeptides. In some embodiments, the RNA-guided molecule further comprises one or more RNA-binding polypeptides. In some embodiments, the RNA-guided molecule is covalently linked to the one or more RNA-binding polypeptides.

In some embodiments, the target nucleic acid in the genome is non-coding DNA at or adjacent to one or more target genes.

In some embodiments, the non-coding DNA includes cis-regulatory elements (CREs).

In some embodiments, expression of the one or more target genes are regulated by the one or more effector proteins or fragments thereof.

In some embodiments, the expression of the one or more target genes is activated or repressed.

In some embodiments, the one or more effector proteins or fragments thereof are covalently linked with one or more RNA-binding polypeptides.

In some embodiments, the targeted therapeutic further comprises: 1) a gene sequence encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or 2) a gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or 3) a gene sequence encoding a third RNA molecule, wherein the third RNA molecule comprises one or more RNA aptamers and wherein the third RNA molecule binds to the one or more RNA-binding polypeptides covalently linked to two or more RNA-binding polypeptides.

In some embodiments, the targeted therapeutic expresses 1) one or more effector proteins or fragments thereof covalently linked to one or more RNA-binding polypeptides; 2) one or more RNA-guided molecules covalently linked to one or more RNA-binding polypeptides and one or more guide RNAs (gRNA); and 3) one or more RNA molecules, wherein the one or more RNA molecules binds to a) one or more RNA-guided molecules and one or more effector proteins or fragments thereof; b) one or more effector proteins or fragments thereof; and/or c) one or more RNA-guided molecules.

In some embodiments, the one or more RNA-guided molecules is guided to the target nucleic acid in the genome by the one or more guide RNAs (gRNA); the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; and the one or more effector proteins or fragments thereof regulate the target nucleic acid.

In some embodiments, the target nucleic acid is associated with disease.

In some embodiments, the one or more effector proteins or fragments thereof are selected from the group consisting of: transcription factors, DNA modification enzymes and cofactors, and histone modifying enzymes and cofactors, and epigenetic regulators.

In some embodiments, one or more effector proteins or fragments thereof are an entire protein, a fusion protein, an effector domain of the effector protein, or covalently linked effector domains from one or more effector proteins or fragments thereof, or a combination thereof.

In some embodiments, the components of the targeted therapeutic are designed based on 3D structural modeling.

In some embodiments, the targeted therapeutic is inducible. In some embodiments, the subject is a mammal. In some embodiments, the subject is human.

In some embodiments, the targeted therapeutic is present in a composition. In some embodiments, the composition comprises a pharmaceutical composition.

In some embodiments, the pharmaceutical composition comprises a lipid formulation. In some embodiments, the lipid formulation comprises one or more cationic lipids, non-cationic lipids, and/or PEG-modified lipids, or a combination thereof. In some embodiments, the pharmaceutical composition comprises a lipid nanoparticle.

In some embodiments, the targeted therapeutic/lipid particle is administered intranasally, intramuscularly, or intravenously.

### BRIEF SUMMARY OF THE DRAWINGS

FIG. 1 is a diagram demonstrating the present platform for novel design of genomic medicines. The modular system is designed for efficiently optimizing a macromolecular assembly to modulate gene expression at a specific genomic locus.

FIG. 2A shows the structure and sequence of a RNA aptamer. The single stranded RNA aptamer contains two binding sites of a RNA binding polypeptide and the atomic model of the RNA aptamer that was construed based on x-ray structure of the RNA binding polypeptide-RNA complex.

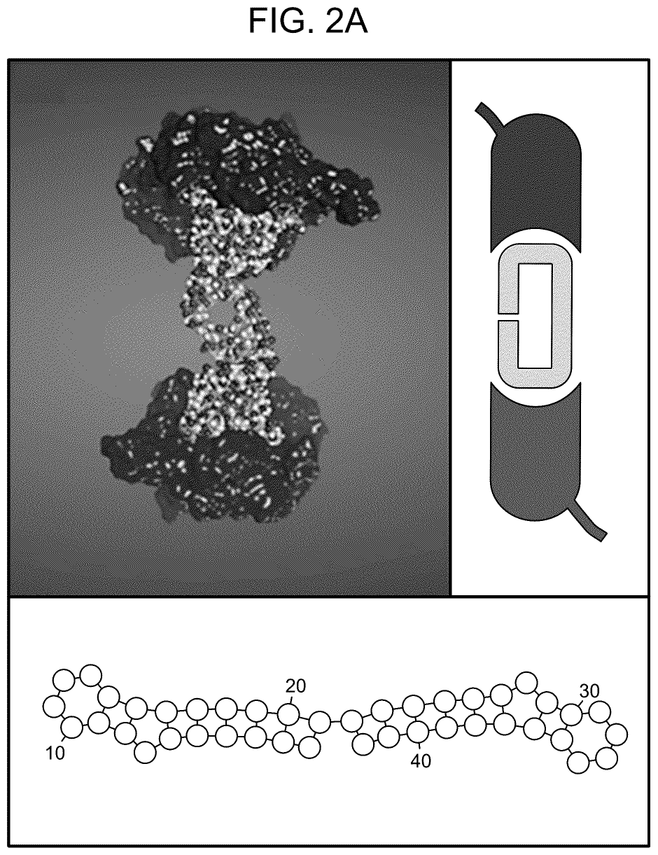

FIG. 2B shows the structure and sequence of a second RNA aptamer. The second single stranded RNA template contains two binding sites of a second RNA binding polypeptide and the atomic model of second RNA aptamer that was construed based on x-ray structure of the second RNA binding polypeptide-RNA complex.

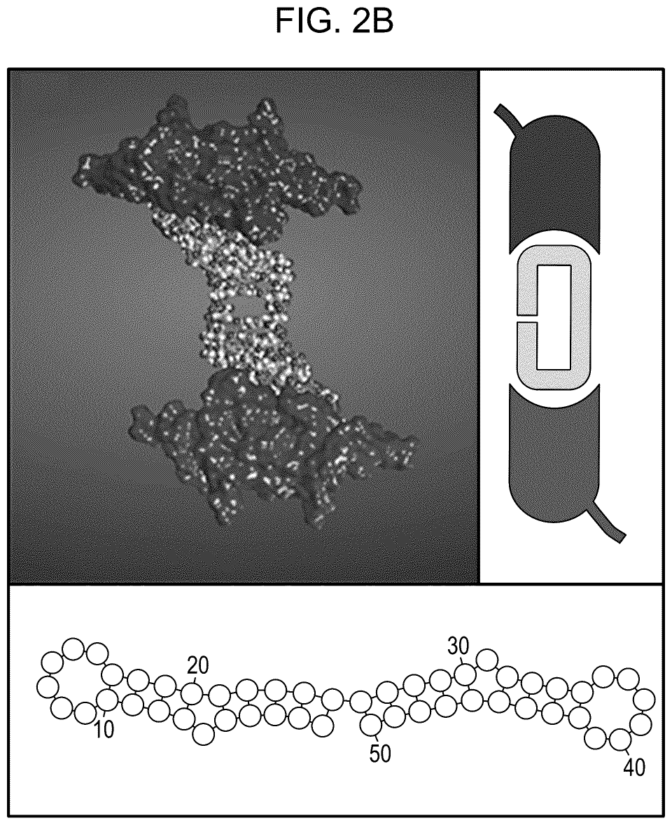

FIG. 3 illustrates a schematic representation of a sgRNA.

FIG. 4 is an atomic model of a promotor region. dCas9 is positioned at a site in the promoter region.

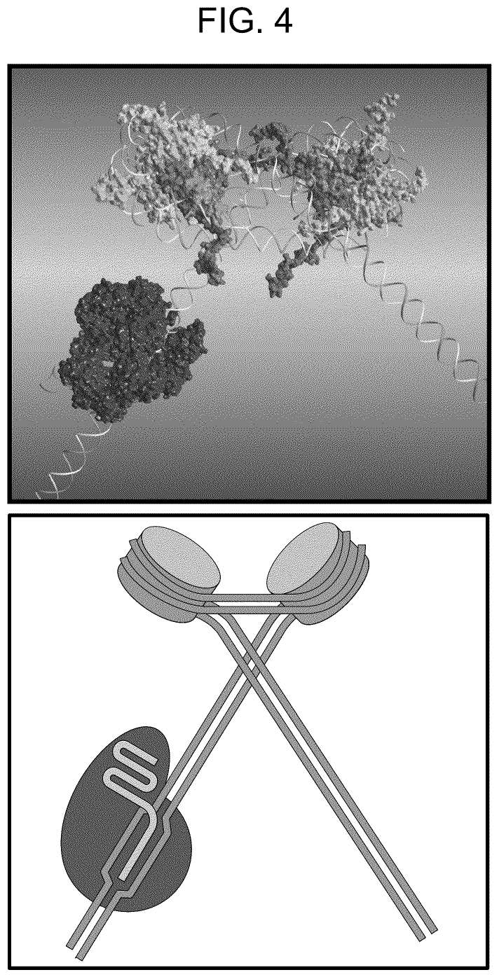

FIG. 5 displays linking two dCas9 enzymes using an RNA aptamer binding to a RNA binding polypeptide fused at both dCas9 enzymes.

FIG. 6 shows molecular model of a promoter region with dCas9 bound at the guide-directed DNA site and a repressor bound at its recognition site. These fusion proteins are cross-linked by an RNA aptamer to the RNA binding polypeptides fused to each component of the macromolecular assembly, i.e., dCas9 and the repressor. Histone proteins in the two nucleosome cores are shown.

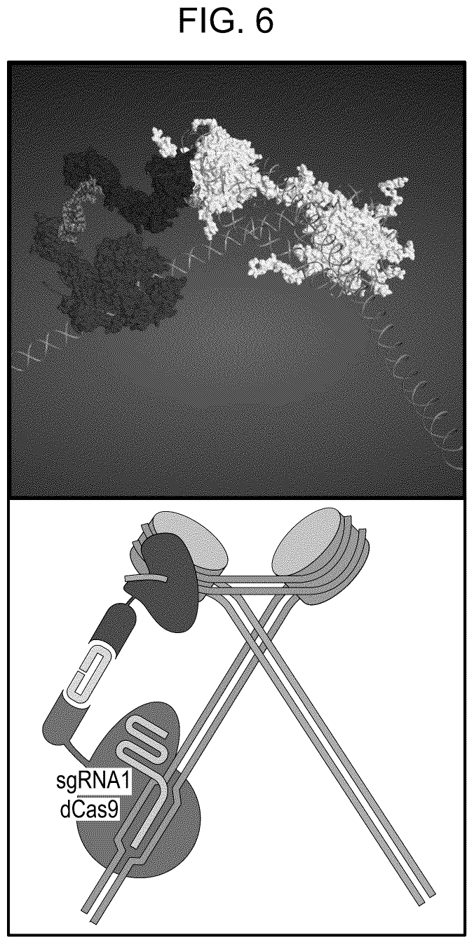

FIG. 7 shows a model of dCas9 linked to a dimer methyltransferases, e.g., methyltransferases specific for transferring methyl groups to specific CpG structures in DNA. Effector protein structures are based on the crystal structure of a methyltransferases dimer in a complex with DNA containing two CpG sites.

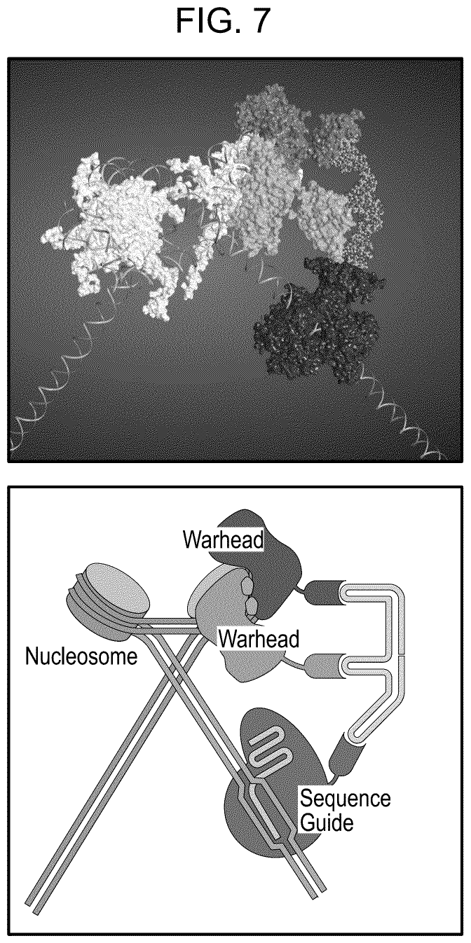

FIG. 8 demonstrates an atomic model of the proposed macromolecular assembly designed for epigenetic editing of histones in a promoter region. dCas9-RNS binding polypeptide binds to demethylase/repressor-RNA binding polypeptide through an RNA aptamer which incorporates three binding sites of the RNA-binding polypeptide. The repressor subunit binds the nucleosome.

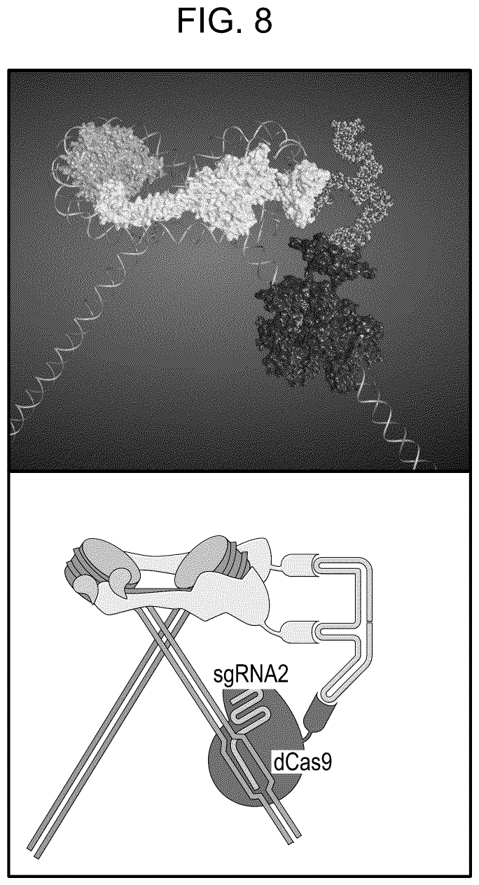

FIG. 9 displays the use of coupled dCas9 using an RNA aptamer at a locus. The histone deacetylate-repressor fusion protein, where the effector protein is fused to an RNA binding polypeptide for linkage to the two dCas9 proteins through the a second RNA aptamer.

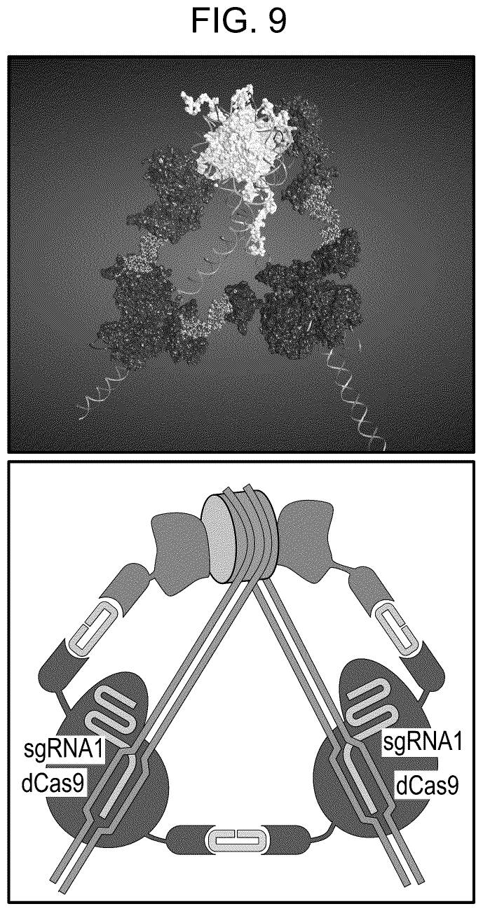

FIG. 10 shows proposed initial toolkit of dCas9-RNA binding polypeptide fusion proteins and effector fusion proteins (as described herein) for modular multiplexed use in gene repression and epigenetic editing.

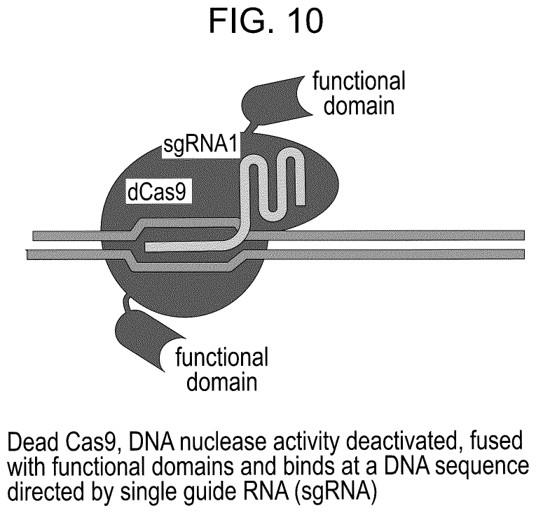

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure relates to novel genomic therapeutics (e.g., targeted therapeutics) for treatment of diseases, particularly those untreatable with traditional drugs such as small molecules and antibodies. The genomic therapeutic discussed herein is a targeted therapeutic designed specifically for targeting to a specific locus in a human genome and modulating a genetic process, e.g., gene expression. Such modulation can correct a genetic defect occurred at the target locus. The targeted therapeutic can be flexibly adjusted for a specific locus, a specific gene or set of genes, a disease or disorder, a subpopulation, and an individual.

The present targeted therapeutic comprises a modular set of self-assembling macromolecular machines comprising a scaffold and effector proteins (e.g., effector proteins, effector domains, or fragments thereof). The assembled macromolecular complex is designed to regulate gene expression at a genetic region (locus) through steric interference and/or remodeling the locus to transiently or more permanently affect its ability to produce RNA transcripts. Such modulation of gene expression consequently increases or decreases or changes the expression level of a gene or set of genes associated with a disease or disorder.

The design of the present targeted therapeutic integrates the principles of structure-guided drug design and are thus built for a target locus in a particular cell type, and in a particular condition. In particular, the selection and design of the macromolecular machines (i.e., effector proteins) of the targeted therapeutic are guided by three-dimensional (3D) models which incorporate available, genetic sequence information, chromatin conformation and epigenetic information at the target locus, but not limited to such information.

The present platform is a ground-breaking combination and integration of tools for protein and nucleic acid structural biology with genomics, molecular biology, bioinformatics, statistical genetics and machine learning. The novel technologies expand the drug discovery platform and revolutionize therapeutics in areas where there have never been effective and safe treatments.

The engineering platform could construct and integrate complex transcriptional programs, chromatin and other epigenetic programs for therapeutic applications. The present platforms and tools would also provide the ability to engineer more sophisticated programs for therapeutic or biotechnological applications.

In accordance, these novel therapeutics and programs provide safe, effective and personalized medical treatments for novel infectious agents (e. g., SARS-COV-2) as well as a vast array of human diseases and disabilities, underscoring a critical need for technological innovation in discovering new therapeutics.

### I. COMPOSITIONS OF THE PRESENT DISCLOSURE

### Targeted Therapeutic for Regulating Gene Expression

The present disclosure provides a targeted therapeutic that acts as a genomic therapeutic for disease treatment and prevention. The targeted therapeutic uses CRISPR-dCas9 technologies to direct a transcriptional regulation machinery, i.e., a modular set of effector proteins, to a specific genetic region (a locus), wherein the effector proteins self-assemble to form a macromolecular complex to modulate the genetic activities at the genetic region. The targeted therapeutic directed to a specific locus is rationally designed, in which the effector proteins are selected in alignment with a desired genetic regulation such as activation or repression of expression of a gene or set of genes at or near that locus. The ability to artificially control gene expression is essential to the study of gene function. In some cases. the target locus, and/or the gene(s) have been identified previously to be associated with a disease or disorder. In this context, the targeted therapeutic acts as a genomic medicine to treat or prevent the disease or disorder. For example, the targeted therapeutic can correct a genetic defect such as a DNA mutation causing loss-of-function or gain-of-function, chromosomal translocation and epigenetic change.

The machinery responsible for gene transcription is tightly regulated and involves a network of DNA-protein and protein-protein interactions and structural changes. Gene transcription is a dynamic process spatially and temporally. At a specific locus, the complex interactions between DNA sequence (e.g., local CpG density), DNA chemical modifications (e.g., DNA methylation), chromatin structure (e.g., nucleosome positioning), histone modifications, other epigenetic regulators as well as transcription factors control gene transcription. It is necessary to considers those players when designing a targeted therapeutic to modulate the expression of a gene or set of genes at a specific locus.

Transcription factors (TFs) are important players in controlling gene transcription. A list of human transcription factors is reviewed by Lambert et al., (The human transcription factors, Cell, 2018, 172(4): 650-665; the contents of which are incorporated herein by reference in their entirety). In accordance with the present disclosure, effector proteins of the system may include TFs that bind to DNA sequence to regulate gene transcription including transcription activators, transcription repressors, coactivators, corepressors, and transcription modulators.

Epigenetic modifications of DNA sequences and histone proteins also play an important role in regulating gene expression. DNA methylation is one of the main epigenetic modifications. It controls gene expression through altering chromosomal structure, DNA conformation, DNA stability and the interaction between DNA and protein (e.g., TF). The DNA methylation pattern of a sequence is a mechanism of cellular memory and carries important information about the programming of gene expression. DNA methylation has the potential to alter gene expression through both direct and indirect mechanisms. DNA methylation in gene promoters can impede the transcriptional machinery from accessing DNA and initiating transcription. Alternatively, DNA methylation can regulate gene expression by altering chromatin structure. A group of methyl-CpG-binding proteins can bind to methylated DNA and mediate interactions between DNA methylation and histone modifications, producing a repressive chromatin structure.

The enzymes and cofactors that play a role in maintenance of DNA methylation state include DNA methyltransferases, which catalyze the addition of a methyl group to the cytosine base or maintain DNA methylation.

DNA methylation changes have been characterized in a wide variety of human diseases, including cancers metabolic disorders, cardiovascular diseases, and imprinting diseases such as Angelman syndromes.

Histone modification patterns are numerous and complex. It is known that histone modifications can substantially influence gene transcription, e.g., by impacting chromatin structure and modulating access to the DNA. Histone modifications include acetylation, methylation, ubiquitylation, phosphorylation, sumoylation, ribosylation and citrullination. Histone modifications are reversible and dynamically changing from activating to repressing patterns. The unstructured N-termini of histones (called histone tails protruding from the nucleosome core particles) are particularly highly modified, which govern the interactions between the nucleosome and other chromosomal proteins. Modifications within the histone core often affect the interactions between the nucleosome and the DNA, which can not only directly regulate transcription, but also influence processes such as DNA repair, replication, stemness, and changes in cell state.

Enzymes and cofactors involved in histone modifications include histone acetyltransferase, histone deacetylases, histone methyltransferases, and histone demethylase.

Other epigenetic regulators that influence chromatin conformation, nucleosome positions also play an important in controlling gene transcription. These epigenetic effectors are important machines of the present targeted therapeutic. A set of effector proteins specific to a locus are designed and their structural relationship at the target site are examined for gene transcription. In general, the system comprises the CRISPR-dCas9 positioning system, in combination with a programmed modular set of effector proteins that are pre-selected according to the purpose of gene expression modulation. The selected effector proteins use RNA aptamers and their cognate RNA-binding proteins for self-assembling.

In accordance, the present disclosure provides a platform for designing a targeted therapeutic to act as a genomic therapeutic that binds to a specific locus associated with a disease or disorder. The design process comprises identification of the genetic locus associated with a disease or disorder, decoding the effector proteins that are required to achieve a particular gene regulation at that genetic locus, and testing the complex formed by the self-assembled effector proteins and predicting the structural relationship of the complex at the target locus, and measuring their effects on regulation of gene expression. The macromolecular components designed through these processes will specifically assemble at a specific target locus. If the readout of gene expression meets the desired purpose (e.g., repression of an over-expressed gene), the system is designed and developed to form a targeted therapeutic for treating or preventing the disease or disorder.

The present targeted therapeutic may correct a loss-of-function variant that causes a disease or disorder. Alternatively, the present targeted therapeutic may correct a gain-of-function variant that causes a disease or disorder.

### Components of Targeted Therapeutics (Genomic Therapeutics)

In one preferred embodiment, a targeted therapeutic, as a genomic therapeutic, comprises: a) CRISPR-dCas9 system; b) a set of RNA aptamers; and c) a set of effector proteins designed for gene repression or activation. These blocks can be combined and further optimized to form a transcriptional regulation machinery targeting a genetic region of interest, such as a locus associated with a disease or disorder.

### CRISPR-dCas Positioning System

The CRISPR-Cas system such as, but not limited to, the CRISPR-Cas9 (type II clustered, regularly interspaced, short palindromic repeat associated protein 9) system is versatile genome engineering tool that has been widely used for modulating gene expression and functions. The system comprises a nonspecific endonuclease (e.g., Cas9) and a set of programmable sequence specific RNAs which guide Cas to a specific target site in the genome, which are referred to as guide RNAs (gRNAs) or a single-guide RNA (sgRNA). The simplicity of this editing system has led to wide-spread use in many biological areas and generated excitement in gene therapy (e.g., Lino et al., Delivering CRISPR: a review of the challenges and approaches. Drug Deliv., 2018, 25:1234-1257).

The CRISPR guide RNA (gRNA) sequence directs the enzyme to a complementary target site and directly impacts the on-target DNA cleavage efficiency and unintentional off-target binding and cleavage. Each Cas nuclease binds to its target site only in presence of a specific sequence, called PAM (protospacer adjacent motif) on the non-targeted DNA strand. Thus, different regions of the genome can be targeted by different Cas nucleases depending on the locations of the PAM sequences.

As used herein, the term “protospacer adjacent motif (PAM)” refers to a short DNA sequence (usually 2-6 base pairs in length) that follows the DNA region targeted for cleavage by the CRISPR system (e.g., CRISPR-Cas9). The PAM sequence is required for a Cas nuclease to cut and is generally found 3-4 nucleotides downstream from the cut site. For example, the most commonly used Cas9 from Streptococcus pyogenes recognizes the PAM sequence 5′-NGG-3′ (where “N” can be any nucleotide base). Table 1 lists Cas9 orthologues and synthetic Cas9 variants and their corresponding PAM sequences. In Table 1, “N” is any base, “R” is adenine (A) or guanine (G), “Y” is cytosine (C) or thymine (T), “WV” is A or T, “D” is A, G or T, “V” is A, C or G, “B” is C, G or T and “M” is A or C.

TABLE 1
Cas9 orthologues and variants and their PAM sequences

| CRISPR | | PAM Sequence |
|---|---|---|
| Nucleases | Organism Isolated From | (5′ to 3′) |
| SpCas9 | Streptococcus pyogenes | NGG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| St1Cas9 | Streptococcus thermophilus | NNAGAAW |
| St3Cas9 | Streptococcus thermophilus | NGGNG |
| BICas9 | Brevibacillus laterosporus | NNNNCNDD |
| CdCas9 | Corynebacterium diphtheriae | NGG |
| ClCas9 | Campylobacter lari | NNGGG |
| FnCas9 | Francisella novicida | NG |
| nCas9RHA | Mutant E1369R/E1449H/R1556A | YG |
| LiCas9 | Listeria innocua | NGG |
| NcCas9 | Neisseria cinerea | NNNNGTA |
| PICas9 | Parvibaculum lavamentivorans | NNCAT |
| PmCas9 | Pasteurella multocida | GNNNCNNA |
| SmCas9 | Streptococcus mutans | NGG |
| SpaCas9 | Streptococcus pasteurianus | NNGTGA |
| TdCas9 | Treponema denticola | NAAAAN |
| LbCpf1 | Lachnospiraceae bacterium | TTTV |
| AsCpf1 | Acidaminococcus sp. | TTTV |
| Cas12j-1 | BiggiePhage Mutant D371A | NNNNVTTR |
| Cas12j-2 | BiggiePhage Mutant D394A | NNNNNTBN |
| Cas12j-3 | BiggiePhage Mutant D413A | NNNNVTTN |
| CjCas9 | Campylobacter jejuni Mutant | NNNVRYMN |
| | D8A/H559A | |
| Nme2Cas9 | Neisseria meningitidis Mutant | NNNNCCNN |
| | D16A/H588A | |

Because of the simple positioning feature of the CRISPR system, increasing efforts have been made to re-engineer the commonly used CRISPR-Cas9 system to expand its applications in addition to as a gene editing tool, including gene regulation, epigenetic editing, chromatin engineering and imaging etc. These alternative applications are largely dependent on the programmable positioning capacity of catalytically inactive dead Cas9 (dCas9) that lacks the DNA cleavage activity but can still be guided to the target site (Jinek et al., A programmable dual-RNA-guided DNA endonuclease in adaptive bacterial immunity. Science 2012, 337: 816-821).

As used herein, the term “dCas9” means a nuclease deficient (or dead or deactivated) Cas9, a variant of Cas9 protein that has no substantial nuclease activity of the corresponding nuclease activity of wild type Cas9 protein. The endonuclease activity is deactivated by introducing one or more mutations to its endonuclease domains.

The dCas9 protein has been adopted as DNA-binding platform, laying the foundation for a whole range of new applications that need site-specific targeting. Different strategies of complexity and efficacy have been developed to employ CRISPR-dCas9 for directing particular molecular effectors to specific sites in the human genome for gene expression modulation and epigenetic editing. Genetic fusion of different effectors (e.g., effector proteins or their functional domain) to dCas9 and expressing the fusion proteins as single recombinant proteins can directly recruit effectors to specific loci in the genome to precisely modulate gene expression. In this application, Single dCas9-fusion proteins can be targeted to adjacent sites of various genes using multiple different sgRNAs for maximum activation.

Current dCas9-mediated transcriptional regulation systems are generalized systems without customization to a particular locus in the genome. It has been recognized that for long-lasting activation or repression of gene expression in a specific locus, a set of effectors are needed to epigenetically modify and re-configure that specific locus. Furthermore, a single configuration or composition of effectors may not suffice to provide a precision solution to effect the desired modulation of the target genes at a specific locus, given the dynamic and variable nature of chromatin across different cell types and in various disease conditions.

The present disclosure utilizes information of chromatin conformation and epigenetic marks at a specific locus in a condition (e.g., cell type, developmental stage, and a disease condition, etc.) and predicts effectors customized for that specific locus in that condition of interest. The selected effectors are recruited to that specific locus using CRISPR-dCas9 positioning system. In addition to direct effector fusion, additional motifs are incorporated into this basic design to recruit effector proteins. The multiplexed, structure-guided design of the targeted therapeutic provides a useful platform to develop genomic medicines for disease treatment and prevention.

dCas9 and Cas Fusion Proteins

In accordance, a targeted therapeutic of the present disclosure comprises dCas9 fusion proteins.

As used herein, the term “RNA-guided molecule” is a molecule (e.g., dCas9) that is guided by an RNA molecule to a target region in the genome.

As used herein, the term “Cas protein” is a clustered regularly imerspaced short palindromic repeats (CRISPR)-Cas (CRISPR-associated proteins) (e.g., Cas9, Cas12, dCas9, and dCas12, can also include dead Cas and Cas nickase).

As used herein, the term “dCas fusion protein” is a chimeric Cas protein in which the Cas is nuclease dead and covalently linked to one or more heterologous polypeptides.

As used herein, the term “dCas9 fusion protein” is a chimeric dCas9 protein in which dCas9 is covalently linked to one or more heterologous polypeptides. The heterologous polypeptide may include any polypeptide that provides a desired property to the dCas9 protein. For the purpose of this disclosure, the heterologous polypeptides include effector proteins or their functional domains. In the context of the present disclosure, a dCas9 fusion protein can bind and modify (e.g., methylate, demethylate, etc.) a target nucleic acid sequence, and/or modify a protein associated with the target nucleic acid sequence (e.g., methylation, acetylation, etc., of, for example, a histone tail). The effector domain exhibits an activity (e.g., an enzymatic activity such as methyltransferases activity, acetyltransferase activity, kinase activity, ubiquitinating activity, etc.), or a regulatory function (e.g., a transcription activation or repression domain of a transcription factor). In some cases, dCas9 is fused with an entire protein (i.e., effector protein) such as an entire transcription activator or repressor protein. In other cases, dCas9 protein may be fused with one or more scaffold polypeptides which provide binding domains for recruiting multiple macromolecules. The binding domains provide the ability of a subject dCas9 fusion protein to bind to another protein of interest, including but not limited to a DNA or histone modifying protein, a transcription factor or transcription repressor, a recruiting protein, an RNA modification enzyme, an RNA-binding protein, a translation initiation factor, an RNA splicing factor, etc. The recruited macromolecules will assemble to form a multiplexed complex at a genetic locus to regulate gene expression.

As non-limiting examples, dCas9 or dCas may be fused with one or more RNA binding polypeptides or protein domains that mediate the RNA binding.

In some embodiments, the heterologous sequence can provide a tag to the dCas9 or dCas protein, e.g., a green fluorescent protein (GFP), or its variants (e.g., YFP, RFP, CFP, etc.)

In some embodiments, dCas9 or dCas may be fused with a heterologous polypeptide that is a small molecule or drug responsive transcription factor (e.g., a transcription activator or a transcription repressor).

A subject dCas9 or dCas fusion protein may have multiple fused polypeptides and domains in any combination of the above.

In some embodiments, additional motifs are added to the dCas9 or dCas fusion proteins. Additional motifs added to dCas9 or dCas fusion proteins may be used to recruit multiple copies of an effector protein, or multiple effectors simultaneously to increase target gene expression more than the simple dCas9/dCas-effector fusion protein.

dCas9 or dCas fusion proteins can target the fused heterologous polypeptides (e.g., effector domains and/or effector proteins) to virtually any genomic region. dCas9 or dCas fusion proteins can flexibly target a locus or many loci in parallel, by using Cas9 or Cas binding guide RNAs (gRNAs) or a single guide RNA (sgRNA) that recognize complementary target DNA sequences. dCas9 or dCas fusion proteins, together with other components of the present targeted therapeutic, can precisely modulate gene expression in that target locus, or several loci simultaneously.

In addition to commonly used dead Cas9 from Streptococcus pyogenes, a dead nuclease may be derived from other Cas enzymes listed in Table 1. In some embodiments, novel engineered forms of dead Cas9 from Staphylococcus aureus (SaCas9) based on its crystal structure are used as the positioning system of the present platform. Such novel dead Cas9 (SaCas9) is smaller than Cas9 from Staphylococcus pyogenes and well suited for delivery using adeno-associated virus (AAV). sgRNAs

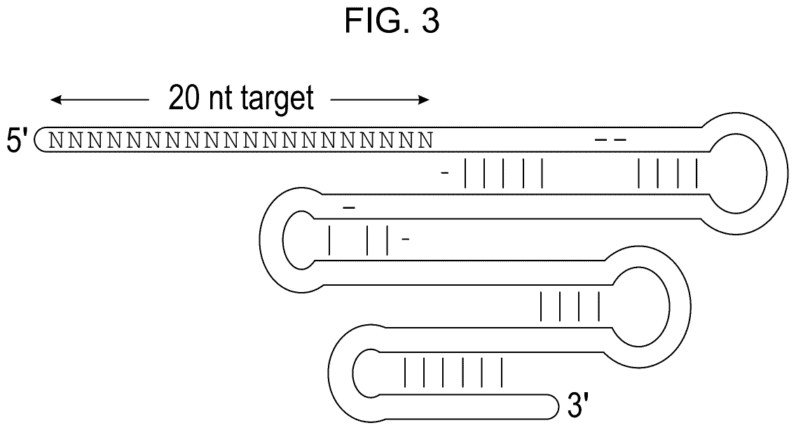

Another key component of the CRISPR-Cas9 system is guided RNAs (gRNAs), which are specific RNA sequences that recognize complementary DNA sequences and direct the non-specific Cas nuclease to the target locus (or loci) containing the complementary DNA sequences for editing. The gRNA is composed of a CRISPR RNA (crRNA), a 17-20 nucleotide sequence complementary to the target DNA sequence, and a tracr RNA that serves as a binding scaffold for the Cas nuclease. The crRNA part of the gRNA can be customized to be specific to any target region in a genome. A single guide RNA (sgRNA) is a single stranded RNA molecule that contains both the custom-designed short crRNA sequence fused to the scaffold tracr RNA sequence. The sgRNA can be synthetically generated or made in vitro or in vivo from a DNA template. A schematic representation of a sgRNA is illustrated in FIG. 3.

The specific guide RNA sequence to a target locus and Cas nuclease can be designed using software tools to optimize the gRNA sequence with minimum off-target effects and maximum on-target efficiency. Any commercially available tools can be used such as the most popular guide RNA design tools Synthego Design Tool, Desktop Genetics and Benchling.

The guide RNA can be made by synthetically generating the sgRNA or by making the sgRNA in vivo or in vitro, starting from a DNA template. In some examples, the sgRNA can be expressed using plasmids. In this method, the sgRNA sequence is cloned into a plasmid vector, which is then introduced into cells. The cells use their normal RNA polymerase enzyme to transcribe the genetic information in the newly introduced DNA to generate the sgRNA. The sgRNA can also be made using in vitro transcription (IVT).

The sgRNA can be extended to include RNA aptamers and other RNA sequences to which RNA binding polypeptides can bind. The RNA aptamers form secondary RNA structures (e.g., hairpins) specifically recognized by RNA binding polypeptides, therefore, serving as the binding sites of RNA binding polypeptides.

In some embodiments, the present targeted therapeutic may comprise a sgRNA that can guide dCas9 protein or a dCas9 fusion protein to a genetic locus associated with a disease or disorder. In other embodiments, the present program platform may comprise a sgRNA that further comprises one or more RNA aptamers. The modified sgRNA not only functions as guide RNA to position dCas9 or dCas9 fusion protein, but also serves as the RNA template to recruit other effector proteins required for the modulation of gene expression in the target genetic locus.

In other embodiments, other CRISPR-dCas systems may be used as the positioning system of the present platform.

### RNA Aptamers

In addition to sgRNAs, the present macromolecular complex comprises a series of novel RNA aptamers to direct self-assembly of the components (e.g., effectors) of the macromolecular complex to specific loci for desired gene regulation. The RNA aptamers complexes the system with added functionality by providing flexibility, modularity and optimization of therapeutic designs based on structural genomics at a specific locus in the genome.

As used herein, the term “RNA aptamers” or “effector template RNA (etRNA)”, used interchangeably, means an RNA molecule that can bind to one or more molecules (e.g., effector proteins, effector domain, or fragments thereof, RNA-guided molecules, or RNA-binding polypeptides). In some embodiments, the RNA aptamer comprises one or more modules capable of binding one or more molecules (e.g., effector proteins, effector domain, or fragments thereof, RNA-guided molecules, or RNA-binding polypeptides). In some embodiments, an RNA molecule comprises a DNA targeting module (e.g., gRNA or sgRNA) and one or more effector protein recruitment modules (e.g., RNA aptamers). The DNA targeting module recognizes specific loci through base-pairing recognition and effector protein recruitment modules attach effector proteins to the loci to execute a specific function at the targeted loci. In some embodiments, the DNA targeting module and effector protein recruitment modules are separate RNA molecules. As a non-limiting example, the DNA targeting module is a CRISPR single guide RNA (sgRNA) that recognizes specific loci and binds to dCas9. The sgRNA is discussed above in detail. Alternatively, the DNA targeting module and effector protein recruitment modules can be combined as a single RNA molecule (e.g., comprising multiple RNA aptamers in a single RNA molecule). For example, a targeted therapeutic comprises an RNA molecule that comprises a sgRNA sequence linked to RNA aptamers that are one or more effector protein recruitment modules and another RNA aptamer that includes different effector protein recruitment modules to attract more and different effector proteins. The combination of different RNA aptamers provide further flexibility and complexity to the engineered assemblies.

In the context of the present disclosure, the RNA aptamer comprises effector protein recruitment module is a binding site for an RNA-binding polypeptide. The RNA aptamer binding site has a specific secondary RNA structure (e.g., hairpin) recognized by its corresponding RNA-binding polypeptide. In some cases, the RNA molecule (e.g., comprising multiple RNA aptamers on a single RNA molecule) comprises a plurality of binding sites for RNA binding polypeptides. The RNA molecule (e.g., comprising multiple RNA aptamers on a single RNA molecule) may further comprise a plurality of stem regions to space the hairpin structures formed by the RNA aptamers. Through protein-RNA recognition, effector proteins covalently linked to the RNA-binding polypeptides are brought together to form a macromolecular complex. In some embodiments, the RNA molecules provide a scaffold for effector proteins at the target sequence in the genome.

Aptamer-protein interaction systems that have been developed for the fluorescence microscopy imaging of single transcripts in living cells can be adopted for designing the effector template RNAs of the present disclosure.

The RNA molecule may combine the sequences of two or more RNA aptamers that bind to RNA-binding polypeptides. In some embodiments, the RNA molecule comprises two or more copies of the same RNA aptamer that bind to the same RNA-binding polypeptides. In other embodiments, the RNA molecule comprises two or more different RNA aptamers that bind to different RNA-binding polypeptides.

As non-limiting examples, a series of single stranded RNA aptamers containing highly specific binding sites for RNA-binding polypeptides may be selected and combined to form one or more RNA molecules for flexible assembly of macromolecular complexes. An RNA molecule comprises RNA aptamers that bind to one, two, three, four, five, six, seven, eight, nine, ten or more RNA binding polypeptides.

More complex effector template RNAs may be construed depending on the requirements for designing a specific targeted therapeutic at a locus. For example, an RNA molecule may comprise several identical or different aptamer-protein pairs to direct construction of very specific macromolecular complexes to optimize modulation of gene expression at a particular disease locus, and also reduce off-target effects. In one non-limiting example, an RNA molecule of the present application may comprise two, three, four, five, six, seven, eight, nine, ten or more binding sites of the same RNA aptamers. In another non-limiting example, an RNA molecule of the present application may comprise two, three, four, five, six, seven, eight, nine, ten or more different RNA aptamers. The multiple aptamer-protein pairs can promote recruitments of two or more effectors which act in concert at a target locus.

In some embodiments, the RNA-binding polypeptide are fused with effector proteins or effector domains of proteins.

An RNA molecule of the present disclosure may further comprise one or more stem regions each of which is in variable length. The stem region may contain at last 5, 6, 7, 8, 9, 10, 11, 12, 13, 14, 15, 16, 17, 18, 19, 20, 21, 22, 23, 24, 25, 26, 27, 28, 29, 30 or more nucleotides. The RNA aptamer hairpins are at either end of the stem region.

In some embodiments, the length of the stem region is customized for a particular locus. The length may depend on the relative distances of the dCas9 (which is determined by selection of the guide sequence) and the location of the desired binding site of the effectors as predicted by building atomic resolution models of the desired macromolecular assembly bound at that locus.

The aptamer-protein interactions are based on naturally occurring high-affinity interactions between specific RNA structure motifs and their RNA binding polypeptides. In the present systems, both the RNA and protein components may be engineered to optimize their sequences for tighter binding. The RNA-binding domain is optimized to minimize the size of the RNA-protein complex.

### Transcriptional Regulation Machinery

In addition to CRISPR-dCas9 positioning system (e.g., RNA-guided molecule) and RNA molecules comprising DNA targeting module and/or effector protein recruiting modules (e.g., RNA aptamers), the present targeted therapeutic comprises a transcriptional regulation machinery. The transcriptional regulatory machinery includes at least one effector protein or polypeptide (e.g., effector protein or effector domain, or a fragment thereof) that play a role in controlling gene expression (e.g., activation and repression). The effector protein or polypeptide can be transcription factors, cofactors, DNA modification enzymes and cofactors, histone modification enzymes and cofactors, other epigenetic factors, and chromatin structure factors and the like, and functional domains thereof.

### Effector Proteins

In some embodiments, the present targeted therapeutic comprises effector proteins that mediate gene expression modulation as well as gene function. Effector proteins include, but are not limited to transcription activators, coactivators, transcription repressors, corepressors, transcription modulators, DNA modification enzymes and cofactors, histone modification enzymes and cofactors, and other epigenetic modifiers.

In some embodiments, effector proteins are transcription factors (TFs) including, but not limited to, transcription activators, coactivators, transcription repressors, corepressors, and transcription modulators. TFs recognize specific DNA sequences to control chromatin and transcription, which interact with other proteins to form a complex system that guides expression of genome.

In some embodiments, effector proteins are epigenetic effectors including DNA modification enzymes and cofactors, histone modification enzymes and cofactors, and other epigenetic modifiers.

As used herein, the term “epigenetic effectors” may include mediators of chromatin remodeling such as DNA modification enzymes and histones and their posttranslational modifications. DNA methylation is commonly seen in the genome. The methylation has been implicated in transcriptional repression, as the promoter regions of silenced genes possess significantly more methylated cytosines in comparison with actively transcribed genes. It has been reported that methylation (e.g., cytosine) of the promoter region may repress gene expression by preventing the binding of specific transcription factors, and/or attract mediators of chromatin remodeling, such as histone-modifying enzymes or other repressors of gene expression.

Histones and their posttranslational modifications have also been implicated in the organization of chromatin structure and regulation of gene transcription. The fundamental building block of chromatin is the nucleosome which consists of DNA spooled around an octamer of histones. Histones generally are classified into H1, H2A, H2B, H3, and H4. Each octamer contains two units of each principal or variant histone H2A, H2B, H3, and H4. The DNA sequence between nucleosomes (i.e., linker DNA) associates with the main form or variants of the linker histone H1. A variety of histone-modifying enzymes can post-translationally modify specific serine, lysine, and arginine residues on the amino-terminal tail of these histones. A specific pattern of the posttranslational modifications on the histones is closely correlated with gene transcription; such correlation results in the histone code. Enzymes have been identified for acetylation, methylation, phosphorylation, ubiquitination, O-GlcNAcylation, sumoylation, ADP-ribosylation, deamination and proline isomerization of histones.

In some embodiments, effector proteins can be histone-modifying enzymes, including but not limited to, histone acetyltransferases (HATs), histone deacetylases (HDACs), histone lysine demethylases (HDMs), histone methyltransferases (HMTs) such as histone-lysine N-methyltransferases and histone-arginine N-methyltransferases.

Histone deacetylases (HDACs) regulate gene expression by removing acetyl groups from lysine residues in histone tails resulting in chromatin condensation (Park & Kim, A short guide to histone deacetylases including recent progress on class II enzymes. Exp. Mol. Med. 2020, 52:204-212).

In accordance with the present disclosure, effector proteins of a targeted therapeutic may be fused with a scaffold protein or polypeptide such as RNA-binding polypeptides that bind to RNA aptamers of the RNA molecule.

### Effector Domains

In some embodiments, effector domains of effector proteins are used as effectors of the present targeted therapeutic. The effector domains are covalently linked to scaffold proteins or polypeptides such as RNA binding polypeptides that bind to RNA aptamers of the RNA molecules presently described.

As used herein, the terms “effector domain” refers to a functional or structural modular of a protein such as a transcription factor (TF) that can serve as a biological effector. The transcriptional effector domain can function in gene expression regulation via their ability to 1) interact with the basal transcriptional machinery and general co-activators and corepressors, 2) interact with other transcription factors to allow cooperative binding, and 3) directly or indirectly recruit histone and chromatin modifying enzymes.

Effector domains can be transactivation domains (TADs) that stimulate transcriptional activation through contacts with general TFs (basal transcription machinery). TADs may be rich in acidic amino acid residues, in glutamine residues, or in proline residues. Effector domains may mediate the interaction of other site-specific factors. Effector domains that can recruit chromatin modifying enzymes including histone-modifying enzymes (e.g., HATs and histone methyltransferases (HMTs)) may mediate both activation and repression.

Effector domains may also include those reside in proteins that can recognize epigenomic marks which can distinguish distinctively modified DNA and histone protein “motifs”. A family of proteins that contain a conserved methyl-CpG binding domain (MBD) binds specifically to methyl-CpG motifs located throughout the genome. In accordance, effector domains can be brought to specific regions of the genome by DNA binding proteins, methyl-CpG binding proteins, or histone binding proteins.

Effector domains may include the catalytic domain (warhead) of an enzyme. As used herein, the term “warhead” is a protein including an effector, an effector domain, or fragments thereof. In some cases, effector domains are the catalytic domains of epigenetic modifying enzymes, e.g., the catalytic domains of the histone acetyltransferases. In other cases, an effector domain is the catalytic domain of a recombinase or nuclease.

Effector domains can be separated from their natural proteins and be engineered to be part of a fusion protein having a DNA binding function (e.g., a heterologous DNA binding domain of another transcription factor), or a fusion protein having a RNA-binding polypeptide (e.g., RNA-binding polypeptides that bind to RNA aptamers). Numerous studies have shown that simply recruiting such effector domains to promoter regions at specific genomic regions can modulate transcription.

In some embodiments, effector proteins or effector domains are fused with RNA-binding polypeptides or the binding domains of the RNA binding polypeptides.

In some embodiments, the effector fusion constructs may include one or more elements of modular designs and multiplexing Which can recruit multiple copies of an effector protein and/or effector domain.

In some embodiments, the effector protein, effector polypeptide, or fragments thereof are expressed endogenously in the cell of interest. In some cases, multiple endogenously expressed effector proteins and/effector domains are recruited to the targeted therapeutic located at the loci of interest.

In some embodiments, RNA aptamers are expressed naturally and bind to the RNA binding polypeptides or the targeted therapeutic as disclosed herein.

### Toolkits and Effector Proteins or Effector Domains “Warheads”

In another aspect of the present disclosure, toolkits and effector proteins (e.g., effector proteins, effector domains, or fragments thereof) designed for gene repression through steric hindrance of transcription, DNA methylation, and/or histone modification are provided. In other cases, toolkits and effector proteins (e.g., effector proteins, effector domains, or fragments thereof) designed for gene activation through steric hindrance of transcription, DNA methylation, and/or histone modification are provided. In addition, the toolkits and warheads can be expanded to include base editing, wherein the effector protein (e.g., effector proteins, effector domains, or fragments thereof) can revert a single nucleotide variant to wild-type to remove the variant and thus replace production of the mutant protein with the wild-type and restore function of a normal protein.

### Programmable Transcriptional Regulation Systems

### Design of Targeted Therapeutics for Regulating Gene Expression

In another aspect, the present disclosure provides design platforms and toolkits for a targeted therapeutic programming a modular set of macromolecular machines to regulate gene expression, particularly designing a targeted therapeutic as a genomic therapeutic for treating and/or preventing a disease or disorder. Such drug discovery platform includes a number of design features that overcome limitations of the current dCas9 mediated systems. The present targeted therapeutic is more amenable for development of precision therapeutic applications. In accordance, the design platform comprises the steps of a) identifying a genetic region that is associated with a disease, b) characterizing the nucleosome pattern of the genetic region, and/or identifying a gene or set of genes associated with the disease in the genetic region, c) selecting a set of effector proteins for modulating the expression of the gene or set of genes in the genetic region and predicting the 3D structure of the self-assembled effector proteins in association with the genetic region, and d) designing the effector proteins of a macromolecular complex assembly that acts as the targeted therapeutic to regulate gene transcription at the genetic region. Optionally the design further comprises a step to evaluate the gene expression regulated by the designed system in cell culture.

### Targeted Genomic Regions

In accordance with the present disclosure, any genomic regions (i.e., loci) can be the targets of the targeted therapeutics. The targeted region may be a coding region, i.e., a gene that encodes a protein. Alternatively, the target region may be a non-coding region in the genome.

In some examples, the targeted therapeutic of the present disclosure targets a coding region, where one or more genes locate. The targeted therapeutic comprises one or more RNA guided molecules, one or more RNA molecule comprising one or more RNA aptamers, and one or more effector proteins or effector domains, or fragments thereof, which assembles into a macromolecular complex through RNA-protein, DNA-RNA, and DNA-protein interactions. The macromolecular complex assembles at the target region and modulates gene activity.

In other examples, the targeted therapeutic of the present disclosure targets a noncoding region. The noncoding region includes regulatory elements for determining when and where genes are turned on and off. In particular, the noncoding sequences provide sites for specialized proteins (e.g., transcription factors (TFs)) to attach and either activate or repress the transcription of genes. The noncoding regions may include introns that locate inside a coding DNA sequence, and regulatory elements such as promoters, enhancers, silencers and insulators. Promoters provides binding sites for the protein machinery that carries out gene transcription. Enhancers are DNA sequences that provide binding sites for proteins that help activate gene transcription. Silencers are DNA sequences that provide binding sites for proteins that repress gene transcription. Insulators provide binding sites for proteins that control transcription in a number of ways. These noncoding regulatory sequences are also called cis-regulatory elements (CREs).

Other noncoding regions in the genome include structural elements of chromosomes such as telomeres with repeated noncoding DNA sequences at the ends of chromosomes, and centromeres composed of satellite DNA sequences. The non-protein coding regions also include DNA sequences that provide instructions and templates for the formation of certain kinds of RNA molecules including transfer RNAs (tRNAs) and ribosomal RNAs (rRNAs), which help assemble protein building blocks (amino acids) into a chain that forms a protein, microRNAs (miRNAs), which are short lengths of RNA that block the process of protein production, and long noncoding RNAs (lncRNAs), which are longer lengths of RNA that have diverse roles in regulating gene activity.

In one preferred embodiment, the targeted therapeutic of the present disclosure targets a transcription start site (TSS) of a gene, or a locus near the transcription start site of the gene, or a downstream regulatory region(s) of the gene. The transcription start site is the location where gene transcription starts at the 5′-end of a gene coding sequence in the genome. The regulatory regions close the TSS of a gene can be analyzed with nucleosome positions used for modeling the promoter region of a gene based on chromatin accessibility data obtained from NucMap browser (Zhao, et al. NucMap: a database of genome-wide nucleosome positioning map across species. Nucleic Acids Res. 2019, 47:D163-D169) and an atomic model of the nucleosomes built using Genome Dashboard (Stolz & Bishop, ICM Web: the interactive chromatin modeling web server. Nucleic Acids Res. 2010, 38:W254-W261; and Li, et al., Genome Dashboards: Framework and Examples. Biophys. J. 2020, 118:2077-2085).

In some embodiments, the targeted therapeutic of the present disclosure may be recruited to introns located within protein-coding genes. Some regulatory elements such as enhancers and silencers may be located in the introns.

In some embodiments, the targeted therapeutic of the present disclosure may be recruited to intergenic regions between protein encoding genes.

In one aspect of the present disclosure, the targeted therapeutic is adapted for transcriptional regulation of gene expression, particularly for treating or preventing a disease or disorder through such regulation. In some embodiments, the genomic target can be a genetic region (e.g., a locus) associated with a disease or disorder.

### Repression of Gene Expression

In some embodiments, the present targeted therapeutic for regulating gene expression is employed for the transcriptional repression of endogenous genes. A toolkit of machine components, including dCas9 fusion proteins, RNA molecules and effector proteins, is built to achieve the goal of repression.

The epigenetic profiles of a genomic region are important for repressing gene expression in the genome. Repressed regions in the genome have different epigenetic marks from regions of actively transcribed genes. Different histones and DNA methyltransferases are required to achieve long-term repression at different loci in different cell types and/or at different developmental stages. Histone posttranslational modifications also represent a versatile set of epigenetic marks involved in not only transcription and the stable maintenance of repressive chromatin, but also in variety of disease conditions. In this regard, DNA methylation at CpG sites and reduction of the number of histone post-translational modifications that reduce the tendency of the nucleosome to form a compact structure is necessary to permanently repress the expression of a gene or a cluster of genes.

A number of studies have incorporated epigenetic effectors to alter DNA methylation and the post-translational modifications of histones for gene expression regulation. In several studies, the dCas9 protein has been fused with DNA methyltransferases to methylate cytosine bases at CpG sites in DNA sequences. This dCas9 system has subsequently evolved to incorporate additional motifs allowing the recruitment of multiple effector proteins to a single target locus, with improved efficiency and reduced off-target activity.

Studies have also reported that gene expression can be repressed using dCas9 fused with histone modifying enzymes (e.g., transcriptional repressors, histone deacetylases, histone methyltransferases, and histone demethylases). It has been shown that trimethylation of lysine 9 and lysine 27 on histone H3 (H3K9me3 and H3K27me3) are associated with repressed chromatin regions.

In accordance, the present targeted therapeutic includes epigenetic effectors to repress gene expression. The epigenetic effectors include but are not limited to DNA methylation enzymes and cofactors, histone modifying enzymes and cofactors, and transcription repressors and corepressors. The repressive effectors are customized for a specific gene or set of genes. In some examples, the target gene(s) may be associated with a disease or disorder.

### Activation of Gene Expression

In other embodiments, the present targeted therapeutic for regulating gene expression is employed for the transcriptional activation of endogenous genes. A toolkit of machine components, including dCas9 or dCas fusion proteins, RNA molecules and effector proteins (e.g., transcription activation protein and transcription activation domains), directed towards activation of genes is built to achieve the goal of gene activation.

In accordance, the present targeted therapeutic includes effectors to activate gene expression. The effectors include but are not limited to transcription activators and coactivators DNA methylation enzymes and cofactors, and histone modifying enzymes and cofactors. The active effectors are customized for a specific gene or set of genes. In some examples, the target gene(s) may be associated with a disease or disorder.

### Other Regulatory Activities

In some embodiments, the present targeted therapeutic may be used to identify and characterize functional regulatory elements.

In some cases, the inducible system may be used to activate or repress gene expression in a dose dependent manner. Dose dependent activation of gene expression has been studied by Chiarella et al. (Dose-dependent activation of gene expression is achieved using CRISPR and small molecules that recruit endogenous chromatin machinery, Nat. Biotech., 2020, 38(1):50-55).

In some examples, dCas9 may be fused to a number of different enzyme functions (effector proteins (e.g., effector proteins, effector domains, or fragments thereof)) in combination to yield the best results. The combination of the effector proteins may vary from locus to locus. In this context, the dCas9-effector systems may be referred to as “multiplexed system”. In some examples, the targeted therapeutic may recruit endogenous chromatin machinery (e.g., gene repressors or gene activators)

### Additional Features of the Targeted Therapeutic

According to the present disclosure, the design features of the present targeted therapeutic include the flexibility to build customized macromolecular machines for a specific locus associated with a specific disease or disorder.

In some embodiments, two-point sequence recognition domain is added to the present system to increased specificity of the system for the target locus by using a combination of dCas9 or dCas with modified transcription factors and/or cooperative binding of multiple linked dCas9 or dCas.

In other embodiments, additional features, such as spatiotemporal control of activity of the components of the present system, are introduced. In this regard, systems for precisely controlling effector recruitment, dCas9-DNA binding activity and/or RNA-protein binding activity in space and/or time are incorporated.

In some embodiments, additional features, such as transgenes, are introduced. The targeted therapeutic can include a transgene to complement a mutation (e.g., mutation, deletion, or insertion of the endogenous gene, or altered gene expression due to uncontrolled gene expression, e.g., over-expression or under-expression). In some embodiments, the protein encoded by the transgene is linked to the targeted therapeutic by RNA aptamer-RNA binding polypeptide binding or by binding of the protein encoded by the transgene to one or more of the components of the targeted therapeutic. In some embodiments, the protein encoded by the transgene does not bind to the targeted therapeutic. In some embodiments, the transgene recruits additional effectors to the targeted therapeutic.

### Inducible

Precise control of the present targeted therapeutic across multiple dimensions such as those of dose, time and space, is critical for its therapeutic applications.

One approach is to control the activity of dCas9 or dCas, dCas9 or dCas fusion proteins and/or effector proteins and constructs of a macromolecular complex to regulate its transcription through an inducible promoter, therefore, making the present targeted therapeutic inducible. Exemplary inducible systems may include, but are limited to, doxycycline-inducible gRNA systems (Aubrey et al., An inducible lentiviral guide RNA platform enables the identification of tumor-essential genes and tumor-promoting mutations in vivo. Cell Rep 2015, 10:1422-1432), Tet-on and Tet-off systems, and other inducible gene expression systems.

Inducible control of the system can also be achieved by light induced or small molecule induced reassembly of dCas9 or dCas, sgRNAs, RNA molecules comprising RNA aptamers and effector proteins of the present targeted therapeutic. In some embodiments, methods for post-translational control of protein function using small molecules can be used to induce the present targeted therapeutic on and off (e.g., Zhang et al., Drug Inducible CRISPR/Cas Systems. Comput. Struct. Biotechnol. J. 2019, 17:1171-1177). In some cases, the inducible system is induced using a molecule administered orally to reversibly turn on and off the therapeutic.

One example of small molecule-controlled system is based on the chemically induced dimerization of split protein fragments, the rapamycin-mediated dimerization of FK506 binding protein 12 (FKBP) and FKBP rapamycin binding domain (FRB) of the mammalian target of rapamycin (mTOR) (Fegan et al., Chemically controlled protein assembly: Techniques and applications. Chem. Rev., 2010, 110:3315-3336: and Zetsche et al., A split-Cas9 architecture for inducible genome editing and transcription modulation. Nat. Biotechnol., 2015, 33:139-142). In this system, the C-terminal fragment of dCas9 is fused to KBP and the N-terminal fragment is fused to the FRB domain. In the absence of the inducer, rapamycin, the split-dCas9 system is inactive. Upon rapamycin addition, the split-dCas9 system is irreversibly activated. The activation of the system is rapamycin dose dependent.

Other orthogonal small-molecule regulators that utilize multiple chemically induced dimerization systems may include, for example, abscisic acid-inducible ABI-PYL1 heterodimerization domains, and gibberellin-inducible GID1-GAI heterodimerization domains (Gao et al., Complex transcriptional modulation with orthogonal and inducible dcas9 regulators. Nat. Methods, 2016, 13:1043-1049; and Miyamoto et al., Rapid and orthogonal logic gating with a gibberellin-induced dimerization system. Nat. Chem. Biol., 2012, 8: 465-470). In these systems, dCas9 and effector proteins (or effector domains) are fused to one of the heterodimerization domains, e.g., ABI or GAI, and PYL1 or GID1, respectively.

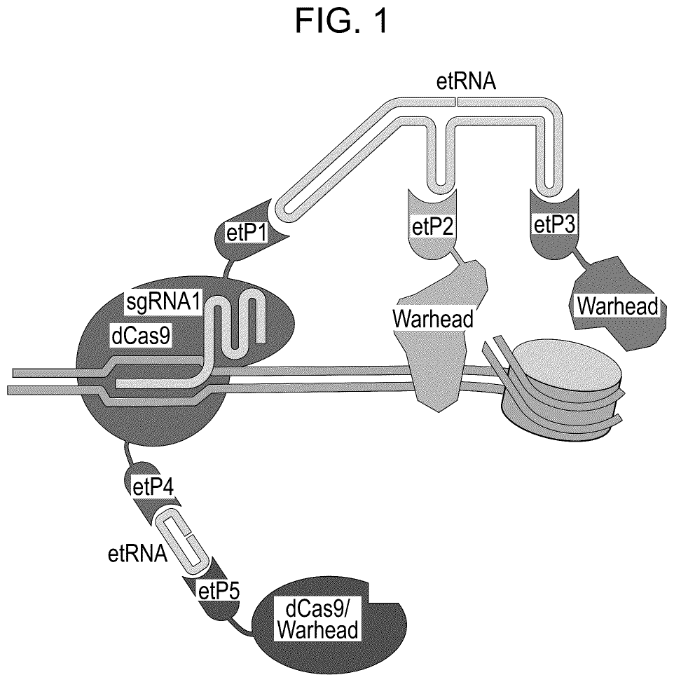

FIG. 1 illustrates an exemplary targeted therapeutic designed according to the present platform. That transcriptional regulation machinery can be used as a targeted therapeutic, to treat and/or present the disease or disorder. This system is designed for efficiently optimizing a macromolecular assembly to modulate gene expression at a specific genomic locus.

### Diseases and Disorders

In some embodiments, the targeted therapeutic regulated gene expression associated with a disease or disorder. In some embodiments, the disease or disease is a cancer, fibrotic disease, infection (e.g., viral infection, bacterial infection, or a fungal infection), respiratory disease or disorder, cardiovascular disease or disorder, intestinal disease or disorder, metabolic disease or disorder, neurological disease or disorder, kidney disease or disorder, liver disease or disorder, systemic disease or disorder, immune-mediated and inflammatory disease or disorder.

### Exemplary Cas Systems

Non-limiting examples of Cas systems that may be used in the programmable systems described herein are provided in Table 2.

TABLE 2
Cas Sequences

| Cas Name | Length |
|---|---|
| dCas12j1 | ATGGCAGATACACCTACCTTGTTCACACAG |
| (SEQ ID | TTTCTGCGGCACCATTTGCCTGGCCAGCGG |
| NO: 1) | TTCAGGAAGGACATCC |
| | TGAAGCAAGCAGGGCGCATACTGGCTAACA |
| | AGGGCGAAGACGCCACTATAGCTTTCCTTA |
| | GAGGCAAGAGCGAGGAGAGTCCTCCCGACT |
| | TTCAGCCACCCGTGAAGTGTCCAATTATTG |
| | CATGTTCCAGACCCCTTACAGAGTGGCCCA |
| | TTTACCAGGCGTCCGTGGCAATCCAAGGTT |
| | ATGTTTACGGGCAGTCCCTGGCCGAGTTCG |
| | AGGCCAGTGACCCCGGCTGCAGCAAGGATG |
| | GCCTGTTAGGATGGTTTGACAAGACCGGTG |
| | TGTGCACCGATTATTTTTCTGTGCAAGGGT |
| | TAAATCTTATTTTTCAGAATGCCCGCAAGC |
| | GCTATATCGGGGTGCAGACTAAAGTAACAA |
| | ACAGAAACGAAAAACGCCACAAGAAACTGA |
| | AGCGGATCAACGCAAAACGGATCGCCGAAG |
| | GTCTGCCCGAGCTTACTAGTGACGAGCCAG |
| | AGAGCGCCCTTGATGAGACGGGTCACCTGA |
| | TAGATCCACCCGGACTGAACACGAATATAT |
| | ATTGCTATCAGCAGGTCTCCCCCAAGCCTC |
| | TGGCCTTGAGTGAGGTTAATCAGTTGCCTA |
| | CAGCTTACGCGGGTTATTCTACCTCAGGGG |
| | ACGACCCTATTCAACCGATGGTAACTAAAG |
| | ACCGTCTGTCCATCTCAAAGGGGCAGCCGG |
| | GATACATTCCTGAACACCAGAGGGCCCTGC |
| | TATCACAGAAAAAACACCGCCGAATGCGGG |
| | GCTATGGGCTCAAAGCTAGAGCTCTGCTCG |
| | TTATAGTGCGGATCCAGGATGACTGGGCCG |
| | TCATTGATCTGCGCTCTCTGCTCAGGAACG |
| | CGTACTGGCGAAGAATTGTGCAGACCAAAG |
| | AGCCGTCCACAATCACCAAACTGCTGAAGC |
| | TGGTGACCGGGGACCCTGTCCTGGATGCCA |
| | CCAGGATGGTAGCTACATTTACATATAAGC |
| | CCGGAATCGTCCAGGTGCGGTCAGCCAAGT |
| | GTTTAAAAAATAAGCAAGGCAGCAAGTTGT |
| | TTTCGGAACGGTACCTGAACGAGACCGTAA |
| | GTGTGACCTCTATTGCACTGGGAAGTAACA |
| | ATTTGGTTGCCGTGGCAACCTACAGATTGG |
| | TGAATGGAAATACTCCCGAACTCCTGCAGC |
| | GCTTCACTTTGCCGTCACACTTAGTCAAAG |
| | ATTTCGAGCGCTACAAGCAGGCACACGACA |
| | CTCTCGAGGATAGCATCCAGAAGACAGCTG |
| | TGGCGTCTCTCCCCCAAGGCCAACAAACTG |
| | AGATACGCATGTGGAGCATGTACGGCTTCA |
| | GGGAAGCCCAGGAGCGAGTGTGCCAGGAAC |
| | TAGGTCTGGCTGACGGCTCCATCCCATGGA |
| | ACGTGATGACGGCTACTTCTACTATTTTGA |
| | CTGATCTCTTTCTAGCAAGGGGGGGCGACC |
| | CAAAGAAATGCATGTTTACCAGCGAGCCAA |
| | AAAAAAAGAAAAACAGCAAGCAGGTCTTAT |
| | ATAAAATTAGAGATAGAGCCTGGGCTAAAA |
| | TGTACAGGACCCTTCTCAGCAAGGAAACGC |
| | GCGAAGCATGGAATAAGGCTCTCTGGGGAC |
| | TTAAACGAGGATCGCCAGATTACGCCAGGC |
| | TTAGCAAGCGAAAGGAGGAGCTCGCCCGGA |
| | GATGTGTTAATTACACAATCTCCACAGCCG |
| | AGAAAAGAGCCCAGTGTGGAAGGACCATTG |
| | TTGCTCTAGAAGACCTCAATATCGGCTTCT |
| | TTCATGGACGGGGGAAGCAGGAGCCAGGAT |
| | GGGTCGGACTATTCACACGTAAGAAAGAGA |
| | ACAGGTGGCTGATGCAAGCCCTCCATAAGG |
| | CCTTCCTGGAACTTGCACATCATCGAGGGT |
| | ATCACGTCATCGAAGTCAATCCCGCGTACA |
| | CCTCTCAGACGTGTCCTGTGTGCAGACACT |
| | GCGACCCAGATAATAGGGATCAACACAACC |
| | GTGAAGCATTCCATTGCATCGGCTGTGGCT |
| | TCCGGGGTAACGCCGACCTGGACGTAGCTA |
| | CCCATAACATTGCTATGGTGGCCATCACCG |
| | GCGAATCACTCAAGAGAGCACGTGGCTCCG |
| | TTGCTTCTAAAACACCTCAGCCTCTGGCGG |
| | CCGAA |
| dCas12j2 | ATGCCAAAACCCGCCGTGGAGTCTGAATTT |
| (SEQ ID | AGTAAGGTTTTGAAGAAGCATTTTCCCGGT |
| NO: 2) | GAACGCTTCCGGAGCAGCTATATGAAGCGC |
| | GGGGGAAAAATCTTAGCCGCTCAGGGGGAA |
| | GAAGCCGTAGTGGCATACCTTCAGGGTAAG |
| | TCTGAGGAGGAGCCCCCAAACTTTCAGCCT |
| | CCCGCGAAGTGCCACGTGGTGACCAAATCT |
| | AGAGATTTTGCTGAATGGCCGATCATGAAG |
| | GCTAGCGAGGCCATTCAAAGGTATATTTAC |
| | GCACTCTCCACTACCGAGAGAGCCGCATGT |
| | AAACCTGGCAAATCATCCGAGAGTCACGCT |
| | GCATGGTTCGCTGCAACCGGCGTTAGCAAT |
| | CACGGATACAGCCATGTTCAGGGACTGAAC |
| | CTGATATTTGACCATACACTGGGACGGTAC |
| | GACGGCGTTTTGAAGAAGGTGCAGCTGCGC |
| | AATGAGAAAGCACGAGCCAGGCTGGAGTCT |
| | ATCAACGCCTCCCGGGCCGACGAAGGCCTT |
| | CCGGAAATTAAGGCCGAGGAAGAGGAAGTG |
| | GCCACCAATGAAACAGGACATCTGCTCCAG |
| | CCACCCGGTATTAACCCTAGTTTTTATGTG |
| | TACCAGACAATCTCACCCCAGGCCTATCGG |
| | CCGAGGGACGAAATCGTGCTGCCACCTGAA |
| | TATGCGGGATATGTAAGAGATCCTAACGCA |
| | CCAATACCACTTGGAGTGGTGCGGAATAGG |
| | TGCGATATCCAGAAGGGCTGTCCCGGCTAC |
| | ATACCGGAGTGGCAACGCGAAGCGGGCACC |
| | GCAATCAGCCCAAAGACGGGAAAGGCCGTC |
| | ACGGTGCCAGGGCTTAGCCCTAAAAAGAAT |
| | AAAAGAATGAGGCGGTATTGGAGGAGTGAG |
| | AAAGAGAAGGCTCAGGACGCACTTTTGGTC |
| | ACAGTCCGGATTGGTACAGACTGGGTGGTC |
| | ATCGATGTACGTGGATTGCTGCGTAATGCC |
| | AGATGGAGAACAATAGCGCCAAAGGATATC |
| | TCCTTGAATGCTCTCTTGGATTTATTTACG |
| | GGGGATCCAGTTATTGATGTTAGAAGAAAC |
| | ATAGTGACCTTTACCTATACTCTTGATGCC |
| | TGCGGGACTTACGCCAGGAAGTGGACACTG |
| | AAGGGCAAACAGACAAAAGCAACTCTCGAC |
| | AAGCTGACCGCTACGCAAACCGTCGCTTTA |
| | GTGGCTATTGCCCTGGGCCAGACCAACCCC |
| | ATTAGTGCCGGAATCAGCAGAGTCACACAA |
| | GAAAACGGCGCGCTACAATGCGAGCCTCTC |
| | GACCGCTTCACTCTGCCTGATGACTTACTG |
| | AAAGACATTTCCGCTTACCGAATCGCCTGG |
| | GACCGCAATGAAGAAGAGCTCCGAGCCCGA |
| | TCTGTGGAGGCCCTGCCAGAGGCACAGCAA |
| | GCAGAAGTCCGAGCGCTGGACGGTGTGTCC |
| | AAGGAGACAGCGAGGACCCAGCTCTGTGCC |
| | GACTTTGGGCTCGATCCCAAACGTCTGCCC |
| | TGGGATAAGATGTCATCCAACACAACCTTC |
| | ATTTCCGAAGCCCTGCTCAGCAATAGTGTC |
| | AGCAGAGACCAGGTGTTCTTCACTCCCGCT |
| | CCGAAGAAAGGTGCGAAAAAGAAGGCCCCA |
| | GTGGAGGTCATGCGCAAGGACCGGACGTGG |
| | GCACGCGCCTACAAACCTAGACTGTCTGTC |
| | GAAGCACAGAAGCTGAAAAATGAGGCACTG |
| | TGGGCTTTAAAGAGGACCTCGCCAGAATAC |
| | CTGAAACTATCACGGCGTAAAGAGGAATTG |
| | TGTCGGAGGTCCATCAACTACGTGATCGAG |
| | AAGACAAGGAGACGGACGCAGTGTCAGATC |
| | GTAATACCCGTGATTGAGGACCTAAACGTG |
| | CGGTTCTTTCACGGATCCGGGAAAAGGCTA |
| | CCCGGGTGGGACAATTTCTTCACCGCTAAG |
| | AAGGAGAATCGATGGTTTATCCAAGGCCTG |
| | CACAAGGCCTTCAGTGACCTCAGAACCCAC |
| | CGGTCTTTCTATGTATTCGAAGTTCGCCCT |
| | GAGCGCACTTCGATTACTTGCCCCAAGTGC |
| | GGCCACTGCGAGGTTGGGAACCGCGACGGC |
| | GAGGCCTTCCAGTGTCTCTCATGTGGTAAA |
| | ACTTGCAACGCTGATCTCGATGTCGCTACC |
| | CATAACCTTACACAGGTAGCACTGACCGGC |
| | AAAACCATGCCTAAACGCGAGGAACCCCGA |
| | GATGCCCAGGGGACTGCTCCCGCACGTAAA |
| | ACTAAGAAGGCTAGCAAATCTAAAGCCCCT |
| | CCTGCCGAGAGGGAGGATCAAACACCTGCT |
| | CAGGAGCCATCACAGACTTCA |
| dCas12j3 | ATGGAAAAAGAGATTACCGAACTGACTAAG |
| (SEQ ID | ATACGTCGCGAGTTCCCAAATAAGAAGTTT |
| NO: 3) | TCGTCAACCGACATGAAGAAAGCCGGCAAG |
| | TTACTCAAGGCAGAGGGCCCTGACGCTGTG |
| | CGTGACTTCCTAAACTCATGCCAGGAGATT |
| | ATCGGCGATTTTAAGCCTCCCGTGAAAACC |
| | AATATAGTGAGTATAAGCCGCCCCTTTGAG |
| | GAATGGCCAGTTAGCATGGTTGGCCGCGCC |
| | ATCCAGGAATACTACTTTTCTTTGACCAAA |
| | GAGGAGCTGGAGAGTGTGCATCCAGGCACA |
| | TCCTCTGAGGACCATAAGAGCTTTTTCAAC |
| | ATTACGGGACTTAGCAACTATAACTACACC |
| | TCCGTGCAAGGTCTAAACCTGATTTTTAAA |
| | AACGCTAAAGCGATTTATGACGGAACGCTC |
| | GTCAAGGCAAATAACAAAAATAAGAAATTA |
| | GAAAAGAAATTTAACGAGATCAATCATAAG |
| | AGAAGCCTGGAGGGCCTTCCAATAATCACA |
| | CCTGACTTCGAAGAGCCATTTGACGAGAAC |
| | GGGCACCTAAACAACCCACCCGGTATTAAC |
| | CGAAATATCTATGGATACCAAGGATGCGCA |
| | GCCAAGGTTTTCGTGCCTAGCAAACACAAG |
| | ATGGTCTCTTTGCCCAAAGAATACGAAGGG |
| | TATAACCGCGATCCCAACCTGAGCCTGGCC |
| | GGTTTTCGCAATAGGTTAGAGATCCCCGAG |
| | GGGGAGCCCGGCCACGTCCCTTGGTTCCAG |
| | CGGATGGATATTCCCGAAGGACAAATCGGA |
| | CACGTGAACAAAATTCAGCGATTTAATTTC |
| | GTCCACGGAAAGAACTCGGGCAAGGTTAAG |
| | TTCTCCGACAAGACTGGTCGGGTGAAGCGA |
| | TACCATCACTCCAAATATAAGGATGCCACC |
| | AAGCCCTACAAATTCCTGGAGGAGTCCAAA |
| | AAAGTCAGCGCCCTGGACAGCATCCTGGCG |
| | ATCATCACAATTGGGGACGACTGGGTAGTT |
| | TTCGATATCCGAGGGTTGTATAGGAATGTG |
| | TTCTACAGAGAGCTTGCTCAGAAGGGCTTG |
| | ACTGCCGTGCAACTGCTGGATCTGTTCACA |
| | GGGGATCCTGTAATTGACCCCAAAAAAGGA |
| | GTAGTTACCTTCTCATACAAGGAAGGGGTC |
| | GTTCCGGTCTTTTCCCAGAAGATTGTGCCG |
| | CGCTTCAAATCCCGGGACACCCTGGAAAAA |
| | CTTACCAGCCAGGGGCCTGTGGCCCTGCTT |
| | TCCGTCGCTCTCGGCCAAAATGAACCGGTG |
| | GCCGCACGCGTGTGTTCATTAAAGAACATC |
| | AATGATAAGATTACTCTGGATAACTCCTGC |
| | AGGATTTCCTTCCTCGATGACTACAAAAAA |
| | CAGATTAAGGACTATCGAGACAGCCTCGAT |
| | GAGCTGGAGATCAAGATACGGTTGGAAGCC |
| | ATTAATTCCCTAGAGACTAACCAGCAGGTG |
| | GAAATCAGGGACCTGGATGTGTTTTCAGCC |
| | GATAGGGCAAAGGCTAATACTGTGGATATG |
| | TTTGACATAGACCCAAACCTTATTAGTTGG |
| | GATAGCATGTCTGATGCACGAGTGAGCACC |
| | CAGATCTCCGATCTCTACCTGAAGAATGGC |
| | GGGGACGAGAGTAGAGTGTATTTCGAGATC |
| | AATAACAAGAGAATTAAGCGGAGTGATTAC |
| | AACATTTCCCAGCTCGTGCGGCCTAAACTC |
| | TCTGATAGCACAAGAAAGAATCTCAACGAC |
| | TCTATATGGAAACTCAAACGGACTAGTGAG |
| | GAGTATCTGAAACTGTCGAAGCGCAAATTA |
| | GAACTCAGCAGAGCTGTGGTGAATTATACT |
| | ATCAGGCAGTCAAAGCTTTTGTCAGGAATC |
| | AATGACATCGTAATAATTCTCGAAGACCTG |
| | GACGTTAAGAAGAAGTTCAACGGCAGAGGC |
| | ATCAGGGATATAGGGTGGGATAATTTTTTC |
| | TCTTCTAGAAAGGAAAACCGGTGGTTTATT |
| | CCCGCGTTTCATAAGGCGTTTTCTGAATTG |
| | TCATCTAACAGGGGGCTTTGCGTGATCGAG |
| | GTAAATCCAGCTTGGACAAGTGCCACCTGT |
| | CCAGACTGTGGTTTCTGTAGTAAAGAAAAT |
| | AGAGATGGTATCAATTTCACATGCCGTAAA |
| | TGTGGAGTCTCTTACCACGCAGACATCGAC |
| | GTCGCCACACTGAACATCGCTAGAGTCGCC |
| | GTGCTGGGTAAGCCGATGAGTGGACCTGCA |
| | GATCGGGAGAGGCTGGGCGATACGAAGAAA |
| | CCTCGTGTCGCCAGGTCCCGCAAAACCATG |
| | AAACGGAAGGACATCTCAAATTCTACGGTT |
| | GAAGCTATGGTAACAGCT |
| dCjCas9 | ATGGCACGAATTCTTGCTTTCGCTATCGGT |
| (SEQ ID | ATCAGCAGTATCGGGTGGGCTTTCTCAGAG |
| NO: 4) | AACGACGAACTCAAAGATTGTGGTGTTCGC |
| | ATATTTACTAAGGTAGAGAATCCGAAAACG |
| | GGAGAGTCCCTCGCCCTGCCGCGCAGGCTG |
| | GCTCGGTCCGCAAGGAAAAGACTGGCCCGT |
| | AGAAAGGCGCGATTGAATCACCTGAAACAT |
| | TTGATCGCCAACGAATTTAAACTCAACTAT |
| | GAAGACTATCAGTCATTCGACGAGAGCCTT |
| | GCCAAAGCTTATAAGGGAAGTCTGATCTCT |
| | CCCTATGAGTTGCGGTTTAGAGCCCTGAAT |
| | GAGCTGCTATCCAAGCAGGATTTTGCTCGC |
| | GTGATCCTGCATATCGCTAAGAGAAGAGGC |
| | TACGATGATATTAAGAATTCAGATGATAAG |
| | GAGAAGGGCGCCATCCTCAAGGCAATCAAA |
| | CAAAATGAAGAGAAGCTGGCAAATTACCAA |
| | AGTGTGGGTGAATATCTGTACAAGGAATAT |
| | TTTCAGAAGTTCAAAGAGAACTCTAAGGAG |
| | TTCACCAACGTAAGGAATAAGAAGGAGTCA |
| | TACGAGAGATGCATTGCACAGTCTTTTCTT |
| | AAAGATGAGCTGAAGTTGATTTTTAAAAAA |
| | CAAAGAGAATTCGGATTCTCATTCTCAAAG |
| | AAATTCGAGGAAGAAGTCCTGTCCGTGGCT |
| | TTTTACAAGAGGGCCTTGAAGGATTTTAGC |
| | CATCTGGTTGGGAATTGCTCTTTTTTTACG |
| | GACGAGAAACGCGCACCTAAGAATTCCCCT |
| | CTGGCCTTCATGTTCGTTGCACTGACTCGA |
| | ATCATCAATCTCCTGAATAATCTCAAAAAC |
| | ACCGAAGGCATCCTGTACACAAAGGACGAT |
| | CTCAACGCCTTATTGAACGAGGTCCTCAAA |
| | AATGGCACACTGACTTATAAACAGACAAAG |
| | AAGCTTCTTGGTCTGTCTGATGATTATGAG |
| | TTCAAAGGGGAGAAAGGGACCTATTTCATC |
| | GAATTCAAAAAGTATAAGGAATTTATTAAG |
| | GCTCTTGGGGAACATAACCTGTCTCAGGAC |
| | GATCTGAACGAAATCGCTAAGGACATAACA |
| | TTAATCAAGGATGAGATCAAACTGAAGAAG |
| | GCACTAGCAAAATACGACTTGAACCAGAAT |
| | CAAATCGACTCTTTAAGTAAACTGGAATTT |
| | AAGGATCACCTTAACATCTCTTTTAAAGCA |
| | CTAAAATTGGTGACTCCACTTATGTTAGAA |
| | GGTAAGAAATACGACGAAGCCTGCAACGAG |
| | CTGAATCTCAAAGTTGCCATCAATGAAGAT |
| | AAGAAAGACTTTCTGCCCGCGTTTAACGAG |
| | ACTTACTACAAGGATGAAGTGACTAACCCA |
| | GTGGTGCTCAGGGCTATCAAGGAATACCGG |
| | AAGGTCCTTAATGCCCTTCTGAAAAAATAT |
| | GGCAAGGTCCACAAGATTAATATAGAGCTG |
| | GCACGCGAGGTCGGCAAAAACCATTCTCAG |
| | CGCGCCAAAATAGAGAAGGAGCAGAACGAG |
| | AACTACAAGGCCAAAAAGGACGCCGAACTC |
| | GAGTGCGAAAAACTTGGACTGAAGATCAAT |
| | TCAAAGAATATTCTCAAGCTCCGTCTTTTT |
| | AAGGAGCAGAAAGAGTTCTGTGCCTACTCG |
| | GGGGAAAAAATTAAAATTAGCGACCTACAG |
| | GACGAAAAGATGCTGGAAATCGACGCTATC |
| | TATCCCTACAGTCGGTCCTTCGACGACTCC |
| | TATATGAACAAAGTACTGGTTTTTACAAAG |
| | CAAAACCAGGAAAAGCTAAATCAGACACCT |
| | TTCGAGGCTTTCGGGAATGACAGCGCCAAA |
| | TGGCAGAAGATAGAGGTGTTGGCTAAGAAC |
| | TTACCTACGAAGAAACAGAAAAGAATACTC |
| | GACAAGAACTATAAAGATAAAGAGCAGAAG |
| | AACTTCAAAGACCGCAACCTTAACGATACA |
| | AGGTACATTGCACGGTTAGTACTGAATTAC |
| | ACAAAGGACTATCTGGATTTCTTGCCCCTC |
| | AGCGATGACGAGAATACCAAGTTAAATGAT |
| | ACGCAGAAGGGCAGTAAGGTCCACGTGGAG |
| | GCTAAAAGTGGCATGCTCACTTCCGCGCTG |
| | AGGCATACCTGGGGATTCTCCGCGAAAGAC |
| | CGTAACAATCACTTACACCACGCAATTGAT |
| | GCCGTGATAATTGCCTATGCCAACAATTCC |
| | ATTGTGAAGGCCTTTAGTGACTTTAAGAAG |
| | GAGCAGGAAAGCAACAGCGCTGAGCTGTAC |
| | GCCAAGAAGATTTCCGAGCTCGATTACAAA |
| | AACAAAAGGAAGTTCTTTGAGCCTTTTAGC |
| | GGCTTTCGGCAAAAGGTCTTGGACAAAATT |
| | GACGAGATTTTCGTCAGCAAGCCCGAAAGA |
| | AAAAAACCCAGCGGGGCACTCCACGAAGAG |
| | ACCTTCCGGAAGGAAGAGGAGTTCTACCAG |
| | AGTTACGGCGGGAAAGAGGGAGTACTGAAG |
| | GCCCTAGAGCTCGGCAAGATACGAAAGGTT |
| | AATGGCAAGATAGTTAAGAACGGAGATATG |
| | TTTCGGGTGGACATCTTTAAGCACAAAAAG |
| | ACCAACAAGTTTTATGCTGTGCCAATCTAC |
| | ACCATGGACTTCGCATTGAAGGTGCTGCCA |
| | AACAAAGCGGTGGCGCGCAGCAAAAAAGGA |
| | GAGATAAAAGATTGGATTCTGATGGACGAG |
| | AACTATGAGTTCTGTTTCTCGCTCTACAAG |
| | GACTCGCTAATCCTGATTCAGACTAAAGAC |
| | ATGCAGGAACCAGAGTTCGTCTACTACAAC |
| | GCTTTTACCTCTTCAACAGTTAGCCTGATC |
| | GTGTCCAAACATGATAATAAGTTCGAAACC |
| | TTGTCTAAGAACCAAAAGATTCTGTTTAAG |
| | AATGCGAATGAAAAAGAAGTGATCGCCAAA |
| | AGCATTGGTATTCAGAACCTGAAGGTCTTC |
| | GAGAAGTATATTGTGTCCGCCCTCGGAGAA |
| | GTGACCAAGGCCGAGTTCCGACAAAGGGAA |
| | GATTTCAAAAAA |
| dCjCas12j2 | ATGCCAAAACCCGCCGTGGAGTCTGAATTT |
| (SEQ ID | AGTAAGGTTTTGAAGAAGCATTTTCCCGGT |
| NO: 5) | GAACGCTTCCGGAGCAGCTATATGAAGCGC |
| | GGGGGAAAAATCTTAGCCGCTCAGGGGGAA |
| | GAAGCCGTAGTGGCATACCTTCAGGGTAAG |
| | TCTGAGGAGGAGCCCCCAAACTTTCAGCCT |
| | CCCGCGAAGTGCCACGTGGTGACCAAATCT |
| | AGAGATTTTGCTGAATGGCCGATCATGAAG |
| | GCTAGCGAGGCCATTCAAAGGTATATTTAC |
| | GCACTCTCCACTACCGAGAGAGCCGCATGT |
| | AAACCTGGCAAATCATCCGAGAGTCACGCT |
| | GCATGGTTCGCTGCAACCGGCGTTAGCAAT |
| | CACGGATACAGCCATGTTCAGGGACTGAAC |
| | CTGATATTTGACCATACACTGGGACGGTAC |
| | GACGGCGTTTTGAAGAAGGTGCAGCTGCGC |
| | AATGAGAAAGCACGAGCCAGGCTGGAGTCT |
| | ATCAACGCCTCCCGGGCCGACGAAGGCCTT |
| | CCGGAAATTAAGGCCGAGGAAGAGGAAGTG |
| | GCCACCAATGAAACAGGACATCTGCTCCAG |
| | CCACCCGGTATTAACCCTAGTTTTTATGTG |
| | TACCAGACAATCTCACCCCAGGCCTATCGG |
| | CCGAGGGACGAAATCGTGCTGCCACCTGAA |
| | TATGCGGGATATGTAAGAGATCCTAACGCA |
| | CCAATACCACTTGGAGTGGTGCGGAATAGG |
| | TGCGATATCCAGAAGGGCTGTCCCGGCTAC |
| | ATACCGGAGTGGCAACGCGAAGCGGGCACC |
| | GCAATCAGCCCAAAGACGGGAAAGGCCGTC |
| | ACGGTGCCAGGGCTTAGCCCTAAAAAGAAT |
| | AAAAGAATGAGGCGGTATTGGAGGAGTGAG |
| | AAAGAGAAGGCTCAGGACGCACTTTTGGTC |
| | ACAGTCCGGATTGGTACAGACTGGGTGGTC |
| | ATCGATGTACGTGGATTGCTGCGTAATGCC |
| | AGATGGAGAACAATAGCGCCAAAGGATATC |
| | TCCTTGAATGCTCTCTTGGATTTATTTACG |
| | GGGGATCCAGTTATTGATGTTAGAAGAAAC |
| | ATAGTGACCTTTACCTATACTCTTGATGCC |
| | TGCGGGACTTACGCCAGGAAGTGGACACTG |
| | AAGGGCAAACAGACAAAAGCAACTCTCGAC |
| | AAGCTGACCGCTACGCAAACCGTCGCTTTA |
| | GTGGCTATTGCCCTGGGCCAGACCAACCCC |
| | ATTAGTGCCGGAATCAGCAGAGTCACACAA |
| | GAAAACGGCGCGCTACAATGCGAGCCTCTC |
| | GACCGCTTCACTCTGCCTGATGACTTACTG |
| | AAAGACATTTCCGCTTACCGAATCGCCTGG |
| | GACCGCAATGAAGAAGAGCTCCGAGCCCGA |
| | TCTGTGGAGGCCCTGCCAGAGGCACAGCAA |
| | GCAGAAGTCCGAGCGCTGGACGGTGTGTCC |
| | AAGGAGACAGCGAGGACCCAGCTCTGTGCC |
| | GACTTTGGGCTCGATCCCAAACGTCTGCCC |
| | TGGGATAAGATGTCATCCAACACAACCTTC |
| | ATTTCCGAAGCCCTGCTCAGCAATAGTGTC |
| | AGCAGAGACCAGGTGTTCTTCACTCCCGCT |
| | CCGAAGAAAGGTGCGAAAAAGAAGGCCCCA |
| | GTGGAGGTCATGCGCAAGGACCGGACGTGG |
| | GCACGCGCCTACAAACCTAGACTGTCTGTC |
| | GAAGCACAGAAGCTGAAAAATGAGGCACTG |
| | TGGGCTTTAAAGAGGACCTCGCCAGAATAC |
| | CTGAAACTATCACGGCGTAAAGAGGAATTG |
| | TGTCGGAGGTCCATCAACTACGTGATCGAG |
| | AAGACAAGGAGACGGACGCAGTGTCAGATC |
| | GTAATACCCGTGATTGAGGACCTAAACGTG |
| | CGGTTCTTTCACGGATCCGGGAAAAGGCTA |
| | CCCGGGTGGGACAATTTCTTCACCGCTAAG |
| | AAGGAGAATCGATGGTTTATCCAAGGCCTG |
| | CACAAGGCCTTCAGTGACCTCAGAACCCAC |
| | CGGTCTTTCTATGTATTCGAAGTTCGCCCT |
| | GAGCGCACTTCGATTACTTGCCCCAAGTGC |
| | GGCCACTGCGAGGTTGGGAACCGCGACGGC |
| | GAGGCCTTCCAGTGTCTCTCATGTGGTAAA |
| | ACTTGCAACGCTGATCTCGATGTCGCTACC |
| | CATAACCTTACACAGGTAGCACTGACCGGC |
| | AAAACCATGCCTAAACGCGAGGAACCCCGA |
| | GATGCCCAGGGGACTGCTCCCGCACGTAAA |
| | ACTAAGAAGGCTAGCAAATCTAAAGCCCCT |
| | CCTGCCGAGAGGGAGGATCAAACACCTGCT |
| | CAGGAGCCATCACAGACTTCA |
| dNme2Cas9 | ATGGCCGCTTTCAAACCCAACCCCATCAAT |
| (SEQ ID | TATATTTTAGGCCTGGCAATCGGTATCGCT |
| NO: 6) | TCCGTGGGATGGGCAATGGTGGAAATCGAT |
| | GAAGAAGAAAATCCTATCCGCCTGATCGAC |
| | TTAGGCGTTCGGGTGTTTGAGCGGGCGGAG |
| | GTTCCGAAAACCGGCGATTCATTGGCAATG |
| | GCCCGCCGCCTCGCACGATCCGTTCGCCGG |
| | CTCACCAGACGAAGAGCTCATCGTCTTCTG |
| | CGGGCACGTCGCCTGCTTAAGCGCGAAGGT |
| | GTCTTACAGGCCGCAGACTTCGATGAGAAC |
| | GGACTCATCAAAAGCCTCCCTAATACTCCG |
| | TGGCAGCTGAGGGCTGCAGCTCTGGATCGA |
| | AAGCTTACCCCACTCGAGTGGAGCGCCGTG |
| | CTGCTACACCTGATTAAGCATAGAGGATAT |
| | CTGTCACAGCGCAAAAACGAAGGGGAGACG |
| | GCTGACAAGGAACTCGGGGCCCTGTTAAAG |
| | GGTGTAGCTAATAACGCACACGCCCTACAG |
| | ACCGGGGACTTCCGCACTCCAGCCGAGCTG |
| | GCCTTGAATAAGTTTGAGAAGGAATCCGGC |
| | CACATAAGAAATCAGCGGGGGGATTACAGT |
| | CACACATTTAGCAGGAAGGACCTTCAAGCA |
| | GAGTTGATCCTGCTCTTCGAGAAGCAGAAG |
| | GAGTTCGGGAACCCACACGTGTCTGGTGGA |
| | CTGAAAGAGGGCATTGAAACATTGCTGATG |
| | ACGCAGAGACCAGCTTTATCTGGAGATGCC |
| | GTCCAGAAGATGCTGGGCCATTGCACCTTT |
| | GAGCCAGCCGAACCCAAAGCTGCAAAGAAC |
| | ACCTATACCGCCGAGCGGTTTATCTGGCTT |
| | ACTAAACTGAACAACCTGCGCATCCTGGAA |
| | CAGGGCTCTGAGCGCCCTCTGACGGACACA |
| | GAAAGGGCGACACTGATGGACGAACCCTAC |
| | CGCAAGAGTAAACTGACATATGCCCAGGCC |
| | AGGAAATTGCTCGGTCTCGAGGATACTGCT |
| | TTTTTTAAGGGCTTACGGTACGGCAAAGAC |
| | AACGCGGAAGCTAGTACTCTCATGGAAATG |
| | AAGGCATACCACGCCATCTCTCGGGCACTA |
| | GAGAAAGAAGGATTGAAAGACAAAAAGTCC |
| | CCCCTCAATCTATCCAGTGAGTTGCAAGAC |
| | GAAATAGGCACCGCCTTCTCTCTCTTTAAG |
| | ACCGACGAGGATATTACCGGCCGACTCAAG |
| | GATCGAGTACAGCCTGAAATTTTGGAGGCC |
| | CTGCTTAAACACATTTCATTCGACAAATTC |
| | GTTCAGATTTCGTTAAAAGCCCTCCGTCGG |
| | ATCGTCCCCCTGATGGAACAGGGGAAGCGT |
| | TATGATGAGGCGTGTGCTGAGATTTATGGG |
| | GACCATTATGGCAAAAAAAACACAGAAGAG |
| | AAAATCTATCTGCCCCCAATCCCAGCAGAT |
| | GAGATCCGCAATCCTGTGGTTCTAAGAGCC |
| | CTGTCTCAAGCCAGAAAAGTGATAAATGGC |
| | GTGGTTAGGCGATACGGTTCTCCAGCCAGG |
| | ATACACATCGAAACTGCTCGCGAAGTGGGG |
| | AAAAGTTTTAAGGATCGAAAAGAGATCGAG |
| | AAAAGGCAGGAGGAAAACAGAAAGGATCGG |
| | GAAAAGGCTGCGGCTAAATTTCGTGAGTAT |
| | TTTCCCAACTTTGTGGGGGAGCCCAAGTCA |
| | AAGGATATCCTGAAACTTAGATTGTATGAA |
| | CAACAGCATGGAAAGTGCTTATACTCCGGT |
| | AAGGAGATCAATCTTGTGAGGTTGAACGAG |
| | AAGGGCTACGTCGAGATTGATGCTGCACTG |
| | CCCTTTTCAAGAACATGGGATGACAGCTTC |
| | AACAATAAGGTTCTGGTGCTGGGCAGCGAG |
| | AATCAGAATAAGGGCAATCAGACACCTTAC |
| | GAATATTTTAATGGAAAGGACAATAGCAGA |
| | GAGTGGCAGGAATTCAAGGCTCGGGTGGAA |
| | ACTTCGCGGTTCCCCAGAAGCAAGAAGCAG |
| | CGGATTCTGCTGCAAAAGTTCGATGAGGAC |
| | GGATTCAAAGAGTGCAACCTCAATGATACT |
| | AGGTACGTAAACAGATTTCTATGTCAGTTC |
| | GTTGCGGATCACATACTGCTCACAGGAAAG |
| | GGCAAACGTAGAGTCTTTGCGAGTAACGGG |
| | CAAATCACAAATCTGCTACGAGGATTCTGG |
| | GGACTCAGGAAGGTGAGGGCCGAGAATGAC |
| | AGGCACCATGCCCTGGACGCAGTTGTGGTG |
| | GCCTGCAGTACAGTCGCGATGCAACAGAAA |
| | ATTACTCGCTTCGTAAGGTACAAGGAGATG |
| | AATGCATTTGATGGTAAGACCATCGACAAG |
| | GAGACTGGTAAAGTACTGCATCAGAAGACC |
| | CATTTCCCTCAGCCTTGGGAATTTTTCGCA |
| | CAAGAGGTGATGATTCGGGTTTTTGGCAAA |
| | CCGGACGGGAAACCTGAGTTCGAGGAGGCC |
| | GATACGCCGGAGAAACTGCGTACCCTTTTG |
| | GCTGAAAAGTTGTCTAGCCGGCCGGAGGCT |
| | GTGCACGAGTACGTGACCCCACTTTTTGTC |
| | AGCAGAGCTCCAAACCGGAAGATGAGCGGC |
| | GCTCACAAAGACACGCTGCGATCCGCTAAA |
| | AGGTTCGTAAAACATAACGAGAAGATTTCC |
| | GTCAAGAGGGTGTGGCTCACAGAAATTAAG |
| | TTGGCCGACCTGGAGAATATGGTCAATTAC |
| | AAGAATGGGCGGGAGATTGAGCTGTACGAA |
| | GCCCTTAAGGCGAGACTGGAGGCTTACGGA |
| | GGTAACGCCAAACAGGCCTTCGATCCTAAA |
| | GACAATCCCTTCTATAAAAAGGGGGGACAA |
| | CTTGTAAAGGCCGTCAGGGTGGAAAAGACC |
| | CAGGAATCCGGGGTTCTCCTTAACAAGAAG |
| | AACGCATACACTATAGCAGACAACGGAGAT |
| | ATGGTGAGAGTCGATGTGTTCTGTAAAGTC |
| | GACAAAAAGGGCAAGAACCAGTACTTTATC |
| | GTCCCTATATACGCCTGGCAGGTGGCCGAG |
| | AATATTCTCCCAGACATCGACTGCAAGGGG |
| | TATCGGATTGACGACTCATATACATTCTGT |
| | TTCTCACTGCATAAGTATGATCTGATAGCC |
| | TTCCAGAAAGACGAAAAGAGCAAAGTGGAA |
| | TTTGCCTACTACATTAACTGCGATAGCTCT |
| | AACGGCCGATTCTATCTGGCCTGGCACGAC |
| | AAAGGGTCCAAGGAACAGCAATTTAGGATC |
| | TCCACCCAGAACTTGGTCCTTATTCAAAAG |
| | TACCAGGTGAACGAGCTCGGAAAAGAAATA |
| | CGCCCTTGTAGATTAAAAAAGAGGCCCCCA |
| | GTGCGC |

### II. FORMULATION AND DELIVERY

### Formulation: Lipid Nanoparticles

In some embodiments, the components of the macromolecular complex may be formulated in lipid nanoparticles (LNPs) (Samaridou et al., Lipid nanoparticles for nucleic acid delivery: Current perspectives. Adv. Drug Deliv. Rev. 2020, doi:10.1016/j.addr.2020.06.002) for delivery to a target cell and/or tissue.

Nanoparticle refers to a structure comprising a lipophilic core surrounded by a hydrophilic phase encapsulating the core. The ionic interaction resulting from the different lipophilic and hydrophilic components of the nanoparticle generates independent and observable physical characteristics. Lipid nanoparticles are useful vehicles for gene therapy.

In some embodiments, the compositions of the present disclosure are formulated in lipid nanoparticles to facilitate delivery to the target cell and tissue. Lipid nanoparticles may comprise one or more cationic lipids, non-cationic lipids, and/or PEG-modified lipids. For example, lipid nanoparticles may comprise cationic lipids such as CI 2-200, DOTMA, DOPE, DOGS, DOSPA, DOTAP, DDAB, DODMA, DLinDMA, DODAC, DMRIE, CLinDMA, CpLinDMA, DMOBA, DOcarbDAP, DLinDAP, DLincarbDAP, DLinCDAP, DLin-DMA, and DLin-K-XTC2-DMA. Suitable cationic lipids may also include the dialkylamino-based, imidazole-based, and guanidinium-based lipids. Lipid nanoparticles may comprise non-cationic lipids. Non-cationic lipids refer to any neutral, zwitterionic or anionic lipids. As used herein, the phrase “anionic lipid” refers to any of a number of lipid species that carry a net negative charge at a selected H, such as physiological pH. Suitable non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Suitable PEG-modified lipids include, but are not limited to, a polyethylene glycol chain of up to 5 kDa in length covalently attached to a lipid with alkyl chain(s) of C6-C20 length.

In other cases, the compositions of the present disclosure are formulated in polymer-liposomes. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

In some embodiments, lipid nanoparticles are prepared by combining multiple lipids and/or polymers.

In some embodiments, the compositions are formulated in combination with one or more additional nucleic acids, carriers, targeting ligands or stabilizing reagents, or in pharmacological compositions where it is mixed with suitable excipients.

### Delivery: Plasmids and Vectors

In accordance with the present disclosure, the components of the present targeted therapeutic can be delivered to a target cell or tissue with any known methods. The dCas9 or dCas fusion proteins, sgRNAs and RNA aptamers, effector proteins and effector domain constructs of the macromolecular complex may be packaged into AAV vectors for delivery.

In accordance with the present disclosure, fusion proteins of the present system are generated with molecular biology technologies. The dCas9 or dCas fusion proteins and effector proteins, effector domain fusions may be encoded by recombinant nucleic acid molecules. In some embodiments, the nucleic acid molecules are cDNAs.

A protein expression system can be used to generate the protein fusion components. In some embodiments, the cDNA molecules are cloned into retroviral vectors, e.g., AAV vectors.

As used herein, the term “AAV (adeno-associated virus) vector” means an AAV viral particle containing an AAV vector genome. It is meant to include AAV vectors of all serotypes, preferably AAV-1, AAV-2, AAV-4, AAV-5, AAV-6, AAV-7, AAV-8, AAV-9, AAV 10, AAV-11, AAV-12, and combinations thereof. AAV vectors resulting from the combination of different serotypes may be referred to as hybrid AAV vectors. AAV vectors may also include AAV variants of the naturally occurring viral proteins, e.g., one or more capsid proteins.

In some embodiments, components of the present targeted therapeutic may be delivered to a genomic locus using AAV vectors to specific tissues and cells (Eichhoff et al. Nanobody-Enhanced Targeting of AAV Gene Therapy Vectors. Mol. Ther.-Methods Clin. Dev., 2019, 15:211-220).

AAV mediated CRISPR-dCas9 delivery may be desirable in cases where a transient repression of a gene is needed. AAV vectors form episomes, extrachromosomal factors that are not integrated into chromosomes, in the host cell nucleus. In dividing cells, AAV episomes are lost through cell division, thereby terminating AAV induced gene therapies.

### Delivery: Routes of Administration

In accordance with the present disclosure, the components of the present targeted therapeutic are formulated to be delivered to a target cell or tissue by any route of administration known in the art. In some embodiments, the targeted therapeutic is formulated as an aerosol to be delivered directly to lung tissue by inhalation. In some embodiments, the targeted therapeutic is formulated to be administered to a subject by pulmonary delivery. In some embodiments, the targeted therapeutic is formulated to be delivered by intravenous administration. In some embodiments, the targeted therapeutic is formulated to be delivered by subcutaneous administration. In some embodiments, the targeted therapeutic is formulated to be delivered by intramuscularly. In some embodiments, the targeted therapeutic is formulated to be delivered directly to the site of inflammation.

### Computational Modeling for Design of Macromolecular Components to a Target Locus

A genetic locus or loci associated with a disease or disorder will be the target of the present targeted therapeutic for regulating gene expression. The suspected genetic locus (or loci) can be identified by a genomic association study or gathered from any public information. Exemplary genomic association studies include GWASs (Genome-Wide Association Studies) and exome and whole-genome sequencing (WGS) studies. Particularly, the present targeted therapeutic is well-suited to target a genetic locus (or loci) identified from GWASs that fall into noncoding regions in the genome. The non-coding regions in the genome are often associated with the disease phenotype, i.e., typically more than 95% of the loci associated with the disease symptoms fall to the non-coding regions. Other epigenetic and transcriptomic data, e.g., gathered from the publicly available repositories worldwide and the published scientific literature may also be used to assist to build relevant regulatory architectures for genetic loci of interest.

The GWAS is an experimental design used to detect associations between genetic variants and traits in samples from populations. GWASs have uncovered thousands of genetic loci and variants that influence risk for human diseases and disorders, as well as complex human traits.

In some embodiments, the GWAS-identified disease risk loci are further evaluated to identify genes associated with a trait or a disease, because an association between a genetic variant at a genomic locus and a trait (including a clinical condition) is not directly informative with respect to the target gene or the mechanism whereby the variant is associated with phenotypic differences. In general, the loci associated with a disease or disorder identified by GWAS can include multiple genes. For example, in a recent GWAS study for suspect loci associated with chronic obstructive pulmonary disease (COPD), the median number of potentially implicated genes per locus in the 82 loci identified, is four, with a maximum of 17 genes (Sakornsakolpat, et al. Genetic landscape of chronic obstructive pulmonary disease identifies heterogeneous cell-type and phenotype associations. Nat. Genet. 2019, 51:494-505). This situation is the critical bottleneck to using GWAS studies to discover new genetically guided targets for developing new drugs.

The present design platform provides a potential resolution to this bottleneck. The platform probes the genetic locus (or loci) that is suspected to be associated with a disease or disorder (e.g., risk locus from GWAS) directly using a macromolecular complex assembled at that genetic locus.

According to the present platform, a first step for designing macromolecular machines of a transcription targeted therapeutic for a specific target locus is to characterize the nucleosome pattern of the target region using computational modeling. The position of nucleosomes with respect to DNA sequences at a specific site influences gene transcription processes. Nucleosomes restrict the accessibility of DNA sequences to protein factors, such as the transcription machinery. The positions and occupancy of nucleosomes can influence the interactions between transcription factors and differences in gene expression between cell types.

The present platform uses the structural information of effector proteins and/or effector domains to generate 3D computational models of the proposed macromolecular assembly that will be recruited to the target locus. The 3D modeling will ensure that spatial relationships and dimensions are correct for that target locus. Incorporating 3D chromosomal architecture at a specific genetic region for gene regulatory network reconstruction will help reveal key transcription factors and epigenetic modifiers driving the desired regulation (e.g., gene repression). Any publicly available structural information about effector proteins and/or effector domains, for example, but not limited to, protein domains for binding to specific DNA sequences, for promoting methylation/demethylation of nearby promoter/enhancer regions and for modulating the accessibility of genes for transcription through epigenetic modifications of histone proteins can be used in the present platform.

In some embodiments, template structures for homology modeling at the locus of Cas9 and effector domains are obtained from RCSB Protein Data Bank (Berman, et al., The Protein Data Bank. Nucleic Acids Res. 2000, 28:235-242) and assembled using the Molecular Operating Environment (MOE) software (Chemical Computing Group, Quebec, Canada). The 3D structures of proteins, nucleic acids and complex assemblies archived in the Protein Data Bank can be used to assist in the design of the present macromolecular complex.

After the genomic locus with nucleosome positions in the “All-Atom” PDB file produced using Genome Dashboard is imported in MOE. Guide RNAs (sgRNAs) are then selected in the regions directly adjacent to the nucleosomes using Cas-Designer (Park, et al., Cas-Designer: a web-based tool for choice of CRISPR-Cas9 target sites. Bioinformatics, 2015, 31:4014-4016) and checked for off-target potential with Cas-OFFinder (Bae, et al., Cas-OFFinder: a fast and versatile algorithm that searches for potential off-target sites of Cas9 RNA-guided endonucleases. Bioinformatics, 2014, 30:1473-1475). Guides with low off-target potential are selected based on modeling to position epigenetic enzyme domains within reach of targeted histones and CpG sites (FIG. 11).

In some embodiments of the present disclosure, the promotor region is predicted by Eukaryotic Promoter Database EPDNew (Dreos, et al., The eukaryotic promoter database in its 30th year: focus on non-vertebrate organisms. Nucleic Acids Res. 2017, 45: D51-D55).

In some embodiments, the modeling process further includes a step to test the proposed macromolecular complexes for feasibility using advanced molecular modeling by integrating 3D atomic structures of the various components in the setting of the indicated nucleosome positions determined by the best available chromatin accessibility data.

The proposed macromolecular assembly, if the 3D modeling suggests a feasible configuration, is directed to the target locus by sgRNAs, thereby linking the effector proteins to the target locus that will perturb the chromatin structure. The genes affected are readout using RNA-Seq without having to choose a particular gene based on a priori considerations. RNA-Seq (RNA sequencing) is a sequencing technique which uses next-generation sequencing (NGS) to reveal the presence and quality of RNA in a biological sample at a given condition to analyze the cellular transcription profiles.

Computational analysis of RNA-Seq datasets will be processed with a pipeline implementation in a Docker container increasing reproducibility of RNA-Seq analysis (see Zhang et al., “Hot-starting software containers for STAR aligner,” GigaScience 7, 2018, 1-5). Sequences are separated into modules (e.g., Docker containers) and all sequence files will be aligned using STAR aligner.

Another application of the present design platform is alternatively to probe the locus to identify a key gene or a cluster of genes that are associated with a disease or disorder. A targeted therapeutic is then developed that acts more directly upon expression of the key gene(s) by targeting the promoter region(s) of that gene or the set of genes. For example, a specific target gene or a set of genes associated with the cytokine storm in COVID-19 infections can be identified. The gene(s) can serve as the target of the targeted therapeutic of the present disclosure.

Once all the components of a proposed macromolecular complex are defined by remodeling prediction and the assembly is tested for feasibility, the encoding nucleic acid sequences (e.g., cDNAs) of each component of the assembly may be packaged into a protein expression system. In some embodiments, the cDNA molecules are cloned into AAV-based viral vectors.

Accordingly, a targeted therapeutic that is developed using the present design platform acts directly upon that genetic locus. If the target locus includes more than one gene, the process provides potential for composing therapeutic programs that repression and/or activation of more than one gene using the same major components of the therapeutic program.

A key feature of the present computational modeling platform is the ability to incorporate the vast wealth of publicly available data by utilizing cloud-capable hardware-agnostic next-generation sequencing (NGS) pipelines. This incorporation allows efficiently and quickly development of novel genomic medicines for diseases.

In some embodiments, the present design platform may further integrate information about DNA methylation states, nucleosome positions, histone modifications and chromatin looping from public data with new healthy and disease single cell data.

In other embodiments, the present design platform may include designs of a graded series of successively more potent repressors or activators, acting first through competitive inhibition with transcriptional machinery to successively more effective and lasting epigenetic remodeling.

Another flexible design feature of the present design process includes construction of 3D models to atomic resolution of the genetic locus influencing expression of the gene of interest to guide the engineering of a therapeutic assembly.

In addition, the present design process may use differential mapping of the local chromatin interactions with transcription factors and non-coding RNAs in disease and healthy tissues by leveraging technologies such as an engineered ascorbate peroxidase 2 (dCas9-APEX2) system.

By directly probing genetic loci that come from GWAS and/or other genomic association studies of common diseases, novel target locus or loci may be discovered and validated by the present computational modeling. As non-limiting examples, common disease may include COPD, Asthma, Diabetes, Rheumatoid Arthritism, Osteoporosis, Schizophrenia, Psoriasis and cystic fibrosis.

### III. METHODS OF USE

In accordance with the present disclosure, the CRISPR-dCas9 mediated macromolecular complex (e.g., RNA-guided molecule) can be positioned to a genetic locus that is known to be linked to a disease or disorder, thereby modulating gene expression and activity at that locus for treatment and/or prevention of that disease or disorder. In one embodiment, the CRISPR-dCas9 system may modulate several loci simultaneously and execute a programmed sequence of therapies. In one embodiment, the dCas protein is not dCas9 and another Cas related protein.

In some embodiments, the complexes and therapeutics designed through the present platforms can be used for a broad range of indications such as cancers, rare genetic diseases, viral infections, and neurological disorders.

The ability to modify CRISPR-dCas9 targeting provides a great opportunity to create programmable macromolecular machines to target any genetic region in human genome. Accordingly, the genomic therapeutics comprising such targeted therapeutics can be developed to treat and/or prevent diseases associated with any specific site(s) in the genome. As a non-limiting example, the disease is lung disease caused by genetic defects where these programmable macromolecular machines can help millions of patients around the world.

### IV. DEFINITIONS

To more clearly and concisely describe the subject matter of the claimed disclosure, the following definitions are provided for specific terms, which are used in the following description and the appended claims. Throughout the specification, exemplification of specific terms should be considered as non-limiting examples.

As used herein, the term “a targeted therapeutic” means a complex system one or more gene sequences encoding one or more effector proteins, one or more gene sequences encoding one or more RNA-guided molecules (e.g., dCas9 or dCas), and one or more RNA molecules comprising RNA aptamers that are capable of binding DNA and/or small RNA binding polypeptides. The one or more effector proteins and one or more RNA-guide molecules bind to one or more RNA aptamers to form an integrated macromolecular complex to execute a transcription regulatory activity at a specific region (locus) in a genome.

As used herein, the term “locus” refers to the specific physical location of a gene or other DNA sequence (e.g., a DNA polymorphism) on a chromosome.

As used herein, the term “gene” refers to the DNA sequences which encode the amino acids found in the nascent polypeptide as a result of translation of a mRNA molecule. In eukaryotes, the coding region is bounded on the 5′-side by the nucleotide triplet “ATG” which encodes the initiator methionine and on the 3′-side by one of the three triplets which specify stop codons (i.e., TAA, TAG, TGA). Genomic forms of a gene are composed of exons (individual coding regions) and introns (non-coding regions between exons). A gene may also include sequences located on both the 5′- and 3′-end of the sequences which are present on the RNA transcript, which are termed “5′ untranslated regions” or 5′UTR and 3′ untranslated regions (3′UTR) respectively. These sequences are also referred to as “flanking” sequences or regions (these flanking sequences are located 5′ or 3′ to the non-translated sequences present on the mRNA transcript). The 5′-flanking region may contain regulatory sequences such as promoters, enhancers and repressors which control or influence the transcription of the gene. The 3′-flanking region may contain sequences which direct the termination of transcription, post-transcriptional cleavage and polyadenylation.

As used herein, the term “transcription” refers to a process in which the genetic information stored in a DNA sequence of a genome is read by proteins and then transcribed into an RNA molecule (messenger RNA) which is later translated into a function protein. The transcription process is highly impacted by the chromatin structure. The term “expression” refers to the biosynthesis of a gene product, i.e., protein, preferably to the transcription and translation of a nucleotide sequence (e.g., an endogenous gene or a heterologous gene) in a cell.

As used herein, the term “nucleic acid” refers to single-stranded or double-stranded deoxyribonucleic acid (DNA) or ribonucleic acid (RNA) and variants thereof. A nucleic acid molecule may be naturally occurring or synthetic or artificial. In the context of the present disclosure, the term “nucleic acid” is used inter-changeably herein with “nucleotide sequence”, “polynucleotide”, “gene”, “cDNA”, and “mRNA”. A nucleic acid molecule may comprise one or more chemical modifications in the chemical structure of the nucleotides including the base, sugar and/or phosphate. Such modifications can include, for example, 5-position pyrimidine modifications, 8-position purine modifications, modifications at cytosine exocyclic amines, substitution of 5-bromo-uracil, backbone modifications, sugar modification, methylations, unusual base-pairing combinations, nucleotide analogues and the like. Nucleotide analogues may include, but are not limited to, analogues with nonnatural bases, e.g., ionosin and xanthine, nonnatural sugars, e.g., 2′-methoxy ribose, or non-natural phosphodiester linkages, e.g., methyl phosphonates, phosphorothioates and peptides.

The skilled artisan will recognize that the term “RNA molecule” or “ribonucleic acid molecule” or “RNA sequence” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure, the ribofuranosyl ring or in the ribose-phosphate backbone.

As used herein, the term “complementary” refers to two nucleotide sequences which comprise antiparallel nucleotide sequences capable of pairing with one another upon formation of hydrogen bonds between the complementary base residues in the antiparallel nucleotide sequences. For example, the sequence 5′-AGT-3′ is complementary to the sequence 5′-ACT-3′. Complementarity can be “partial” or “complete”. “Partial” complementarity is where one or more nucleic acid bases is not matched according to the base pairing rules. “Complete” complementarity between nucleic acids is where each and every nucleic acid base is matched with another base under the base pairing rules.

As used herein, the term “aptamer” is a nucleic acid species that has been engineered through repeated rounds of in vitro selection or equivalently, SELEX (systematic evolution of ligands by exponential enrichment) to bind to various molecular targets such as small molecules, proteins, peptides, nucleic acids, and even cells, tissues and organisms. Nucleic acid aptamers bind with high affinity to specific targets by folding into complex tertiary structures. A typical nucleic acid aptamer is approximately 10-15 kDa in size (15-45 nucleotides), binds its target with at least nanomolar affinity, and discriminates against closely related targets. An aptamer may contain 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides. A nucleic acid aptamer can naturally fold into specific and stable secondary structures that enable it to bind to a selected target. Nucleic acid aptamers may be RNA aptamers, DNA aptamers, or mixed RNA/DNA aptamers. Aptamers may be single stranded RNAs, DNAs or hybrid DNA/RNA molecules.

As used herein, the term “expression construct” and “DNA construct” as used herein are synonyms, refer to a nucleic acid sequence capable of directing the expression of a fusion protein such as a dCas9 fusion protein and an effector protein fused with small RNA binding polypeptide, in a host cell.

As used herein, the terms “protein” and “polypeptide” are used interchangeably. A protein refers to a polymer of consecutive amino acid residues bonded via peptide bonds. The polymer can be linear, branched or cyclic. In the present disclosure, the terms “gene product” and “gene expression product” is also used to refer to a protein or polypeptide. A polypeptide may include amino acids that are L stereoisomers (the naturally occurring form) or D stereoisomers and may include amino acids other than the common naturally occurring amino acids, such as 0-alanine, ornithine, or methionine sulfoxide, or amino acids modified on one or more α-amino, α-carboxyl, or side-chain, e.g., by appendage of a methyl, formyl, acetyl, glycosyl, phosphoryl, and the like.

As used herein, the term “domain” refers to a unit of a protein or protein complex, comprising a polypeptide subsequence, a complete polypeptide sequence, or a plurality of polypeptide sequences where that unit has a defined function.

As used herein, the term “fused” or “covalently linked” with respect to a protein and/or a nucleic acid, refers to linkage by covalent bonding.

As used herein, the term “vector” means any agent capable of delivering and expressing at least one nucleic acid molecule in a host cell or subject. The vector may be extrachromosomal (e.g., episome) or integrating (for being incorporated into the host chromosomes), autonomously replicating or not, multi or low copy, double-stranded or single-stranded, naked or complexed with other molecules. For example, vectors may be complexed with lipids or polymers to form particulate structures such as liposomes, lipoplexes or lipid nanoparticles. In some cases, vectors may encompass viral vectors obtained from a variety of different viruses, such as retrovirus, adenovirus, adeno-associated virus (AAV), poxvirus, herpes virus, measles virus and foamy virus. Vectors may also include non-viral vectors such as, plasmids, Vector may also be modified to allow preferential targeting to a particular host cell.

As used herein, the term “designed” means non-naturally occurring and/or genetically engineered. The designed macromolecular complex described herein differ from wild-type or naturally occurring macromolecular complex in the components and factors at a particular genetic region/locus.

### V. EQUIVALENTS AND SCOPE

Those skilled in the art will recognize or be able to ascertain using no more than routine experimentation, many equivalents to the specific embodiments in accordance with the disclosure described herein. The scope of the disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### VI. EXAMPLES

### Example 1: Nucleosome Patterns at the Target Locus Close to a Promotor Region and dCas9 Positioning

Nucleosome positions at a target locus containing a transcription start site (TSS) have been identified based on the DNA sequence of the human reference genome and micrococcal nuclease sequencing (MNase-Seq) and visualized based on chromatin accessibility data obtained from NucMap. An atomic model of the nucleosomes at the target locus close the TSS was built using Genome Dashboard.

In an example, the TSS may also bound by the histone core of specific nucleosome. The H3K4me3 and H3K37Ac tracks will also be determined to confirm regulation of gene expression may be regulated partially by histone modifications. The model indicates that epigenetic remodeling of the nucleosome may be necessary, by employing epigenetic editing enzyme-containing effector proteins, in order to remove any activating methyl and acetyl groups from the histones to transition to induce a more compact chromatin state and thereby reduce the gene transcription.

To best optimize positioning CRISPR-dCas9 to the gene promotor region, guide RNAs for Cas9 from Streptococcus pyogenes (SpCas9) (PAM 5′-NGG-3′) and Streptococcus aureus (SaCas9) (PAM 5‘-NNGRRT-’3 (R=A or G) were considered. These were placed in the atomic models at the proposed guide positions. The crystal structure of Cas9 from S. pyogenes (pdb:4008) (Nishimasu, et al., Crystal Structure of Cas9 in Complex with Guide RNA and Target DNA. Cell, 2014, 156:935-949) or S. aureus (pdb:5CZZ) (Nishimasu, et al., Crystal Structure of Staphylococcus aureus Cas9. Cell, 2015, 162:1113-1126) with the guide RNA was added to the atomic model with the proposed guide sequences.

To design guide RNAs for directing dCas9 and fused effectors to the target locus in the promoter region of the gene, the genomic region with nucleosome positions in the “All-Atom” PDB file produced using Genome Dashboard was imported in MOE (Molecular Operating Environment) software for template structures for homology modeling at the target locus. Guide RNAs (sgRNA) were selected in the regions directly adjacent to the nucleosomes using Cas-Designer and examined for off-target potential with Cas-OFFinder. sgRNAs with low off-target potential were selected based on modeling to position epigenetic enzyme domains within reach of targeted histones and CpG sites. FIG. 3 demonstrates a schematic representation of a sgRNA molecule.

Based on this model, a set of effectors are selected to build multiple macromolecular complexes, e.g., a targeted therapeutic for inhibition of the gene transcription. The effectors are designed and combined with the CRISPR-dCas9 positioning system which directs the effectors to the gene promotor region.

### Example 2: Design of Linked dCas9 Using an Effector Template RNA

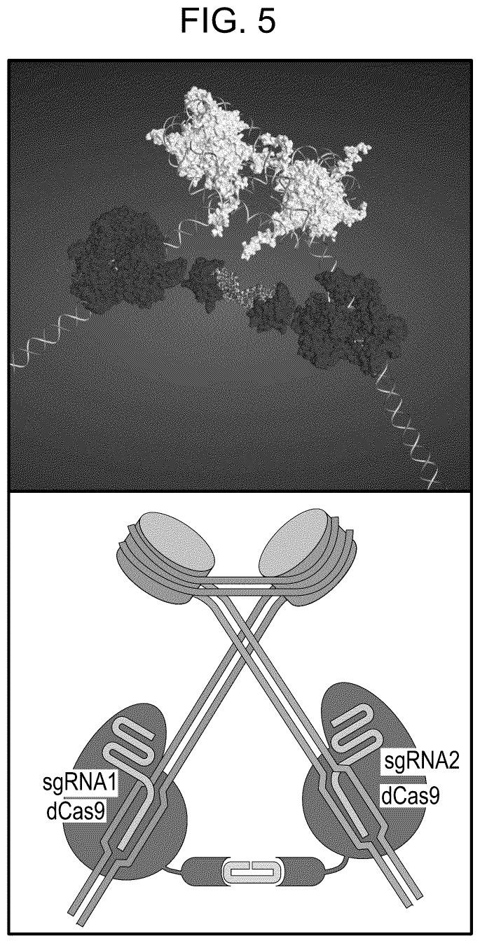

An RNA aptamer (etRNA) was created with two copies of an RNA aptamer that includes the binding site of an RNA binding polypeptide. Using this novel RNA aptamer, two dCas9-RNA binding polypeptide fusion proteins are linked together through the binding of RNA binding polypeptide to the RNA aptamer. The system is used to test for repression of gene expression due to the increased residence time by crosslinking two or more dCas9 proteins directed to independent sites at the gene promoters. The structure modeling of the linked dCas9 enzymes at the promotor region of the gene is shown in FIG. 5.

### Example 3: Plasmids and Cell Transfection

Accordingly, the fusion cDNA constructs encoding dCas9, dCas9 fusion proteins (e.g., dCas9-RNA binding polypeptides) and effector proteins (e.g., effector proteins, effector domain, or fragments thereof) and other effector proteins designed in previous examples, are created following standard protocols of molecular biology. The cDNA constructs are then packaged into AAV vectors for delivery of the constructs into cells.

Human Lung cells (IMR90 cells) (ATCC CCL-186™) are infected with the AAV vectors comprising those cDNA molecules. The expression of the engineered macromolecules, their assembly at the target locus and the function to modulate expression of the target genes are tested and validated.

To show that the engineered macromolecules are expressed in cells, the messenger RNAs of the fusion proteins, the sgRNAs for Cas9 and RNA aptamers, and optionally a transgene, produced in the transfected cells are measured by RNA sequencing. By sequencing the whole transcriptome from both the endogenous chromosomes as well as the introduced AAV episome, any differences in the level of expression of off-target genes relative to untreated cells are accessed.

Expression of the corresponding fusion proteins are detected using commercially available antibodies to domains in the component fusion proteins by Western blot, using recombinant proteins as positive controls.

Binding of the macromolecular assembly to the locus is demonstrated using ChIP-Seq. ChIP-Seq combines chromatin immunoprecipitation with DNA sequencing to identify the binding sites of DNA-associated proteins. If needed, a second occlusion assay is employed, such as ATAC-Seq.

Based on the results of the above assays, a second round of therapeutic design may be needed to improve performance of the macromolecular assembly. Once in vitro optimization is complete, AAV vectors encoding these fusion proteins and RNA molecules are tested in animal models of gene regulation and cytokine storm response.

### Example 4: Pilot Study for Delivery Using Aerosolizable Lipid Nanoparticles (LNPs)

This pilot study is used to test targeted delivery of nucleic acid therapeutics packaged into aerosolizable lipid nanoparticles (LNPs). The LNPs will be engineered to recognize specific cell types through covalent attachment of aptamers known to bind proteins expressed on the surface of the targeted human cells. Higher delivery and expression in the target cell types will provide options to use nucleic acid therapeutics to treat wide range of pulmonary diseases and infections.

### Delivery and Expression Using Untargeted LNPs

Initially, a plasmid encoding for the reporter protein green fluorescent protein (eGFP; λₑₓ=488 nm, λₑₘ=509 nm) will be packaged into an aerosolizable LNP and expressed in human lung cancer cells in a 2D and 3D culture. The LNP transfection will be optimized for the panel of different cell lines by monitoring fluorescence from the eGFP protein as an indication of successful internalization of the LNP with subsequent expression of the nucleic acid payload.

After the human cancer cell study, the transfection of the LNP will be optimized using patient derived micro-organospheroids from lung cancer patients (tumor and adjacent cells, as available) and also measure eGFP fluorescence using flow cytometry as an indication of successful gene expression.

### Delivery and Expression Using Targeted LNPs

A plasmid encoding for the reporter protein will be packaged into an aerosolizable LNP and expressed in human lung cancer cells in a 2D and 3D culture. As a starting point, aptamers will be conjugated to PEG-coated liposomes by adapting the protocol of Kang et al. (A liposome-based nanostructure for aptamer directed delivery. Chem Commun. 46, 249-251 (2010) to conjugate cancer cell specific aptamers to PEG-coated LNPs. The aptamers (Table 14; manufactured and dual-HPLC-purified by Bio-Synthesis, Inc.) will be synthesized using standard bases and phosphodiester linkages with a thiol (HS)-modified 5′ end to react and conjugate with the DSPE-PEG(2000) Maleimide (MalPEG, Avanti Polar Lipids) on the LNP surface and a tetramethylrhodamine (TAMRA) fluorophore-labeled 3′ end, to quantitatively characterize the amount of aptamers linked on the LNP surface and monitor the LNP-cell interaction. Aptamers of similar length with random sequences will also be prepared to control for non-specific binding. The aptamers have been chosen to bind to receptors previously reported to be expressed on lung cancer cells: Nucleolin is widely expressed on the surface of NSCLC cells but not on normal cells. EGFR is a transmembrane protein that is overexpressed in aggressive cancers such as NSCLC and glioblastoma. The Toll-like receptor TLR4 is often present and activated in lung cancer cells. In the CL-4 RNV616 sequence, 2′-O-Methyl RNA nucleotides are represented by ‘m’ and DNA nucleotides are represented by ‘d’.

TABLE 3
DNA Aptamers

| | | | | SEQ | |
|---|---|---|---|---|---|
| | | | Sequence | ID | |
| Name | Target | Length | (5′ to 3′) | NO. | References |
| AS1411 | Nucleolin | 26 | GGTGGTGGTGGTTGT | 7 | Yazdian- |
| | | | GGTGGTGGTGG | | Robati |
| | | | | | (2020) |
| CL-4 | EGFR | 27 | mUmGmCmUmUmUmG | 8 | Wang (2019) |
| RNV616 | | | dAmUmGmUmCmGdA | | |
| | | | mUmUmCmGdAmCdA | | |
| | | | mGmGdAmGmGmC | | |
| GR200 | EGFR | 50 | CGACGCACCATTTGT | 9 | Zavyalova |
| | | | TTAATATGTTTTTTAA | | (2020) |
| | | | TTCCCCTTGTGGTGC | | |
| | | | GTCG | | |
| ApTLR#4 | TLR4 | 59 | GGTGTGCCAATAAAC | 10 | Fernandez |
| FT | | | CATATCGCCGCGTTA | | (2018) |
| | | | GCATGTACTCGGTTG | | |
| | | | GCCCTAAATACGAG | | |
| CTRL-1 | None | 27 | GTTGCATCCCAAATA | 11 | — |
| | | | GAGGACCGCGAT | | |
| CTRL-2 | None | 54 | GTCAAGGCTGCGCGA | 12 | — |
| | | | GGTTGGTCTACAGAA | | |
| | | | ACCACGCTAGTGTAA | | |
| | | | CTTGCCAGT | | |

The aptamers will be pre-validated against a panel of different cell lines before they are used for coupling to the LNP. Cells will be treated with a range of aptamer concentrations (10-500 nM) to measure target-specific binding using flow cytometry by comparison to non-specific binding of a control aptamer of similar length with random sequence.

The LNPs which are formulated for aerosol delivery to the lungs will contain the eGFP plasmid and incorporating MalPEG at a molar ratio of approximately 150 total PEG-conjugated lipids to 1, and this ratio will be adjusted to optimize for desired physicochemical properties as needed. In the end, there will be approximately 250 aptamers bound per LNP.

The conjugation of aptamer to LNPs will be performed by adding aptamer at three different ratios (relative to the amount of MalPEG estimated from phosphate assay: 2.5-fold, 5-fold, and 10-fold excess) to the LNP solution. Prior to use, the aptamer will be diluted at an assay dependent concentration in PBS buffer containing 5 mM MgCl2, and the solution heated at 85° C. for 5 min, incubated for 10 min at room temperature and finally allowed to refold for 15 min at 37° C. The thiol-modified aptamer will then be activated by Tris (2-carboxyethyl) phosphine (TCEP), a reducing reagent, in 100 mM TCEP solution at 4° C. for 30 minutes. The activated aptamer will be mixed with LNP and incubated overnight at 4° C., followed by addition of 2 mM beta mercaptoethanol (BME) to quench the unreacted maleimide group. The aptamer-LNP solution will then be run over a Sephadex column to remove free aptamer, and the phosphate/DNA/TAMRA assays used to characterize LNP composition in the column fractions. Plots of the fractions will be prepared using DNA absorbance in place of FITC fluorescence to monitor the payload of LNPs. The pooled peak fractions containing LNPs with the best composition (lipid, DNA and aptamer) and size will be dialyzed against 500 mL HEPES buffer solution at 4° C. before storing for additional studies.

The uptake of the conjugated aptamer-LNPs will be compared to the LNPs without aptamer into lung cancer cells using flow cytometry and confocal microscopy. The data will be plotted in order to observe a shift in the peaks to the right (indicating more cells with stronger fluorescence) when comparing conjugated vs. unconjugated LNPs at the same concentration, by monitoring the red fluorescence from Aptamer-TAMRA and green fluorescence from eGFP.

1. A method of designing a targeted therapeutic, a method comprising:
a) selecting one or more gene sequences encoding one or more effector proteins or fragments thereof; and
b) selecting a gene sequence encoding one or more RNA-guided molecules and a gene sequence encoding one or more guide RNAs (gRNA), wherein the RNA-guided molecule comprises a dead Cas9 (dCas9) protein, and wherein the gRNA comprises either 1) a crispr RNA (crRNA) and a trans-activating crispr RNA (tracrRNA), or 2) a single guide RNA (sgRNA), wherein the RNA-guided molecule and gRNA interact with a target nucleic acid in the genome, and wherein the one or more effector proteins or fragments thereof are guided by the RNA-guided molecule/gRNA complex to the target nucleic acid in the genome, and wherein the targeted therapeutic comprises one or more nucleic acid constructs.

2.-7. (canceled)

8. The method of claim 1,
1) wherein the gRNA further comprises one or more RNA aptamers, wherein the one or more RNA aptamers are capable of binding one or more RNA-binding polypeptides; and/or
2) the RNA-guided molecule further comprises one or more RNA-binding polypeptides, wherein the RNA-guided molecule is covalently linked to the one or more RNA-binding polypeptides; and/or
3) the target nucleic acid in the genome is non-coding DNA at or adjacent to one or more target genes, wherein the non-coding DNA includes cis-regulatory elements (CREs), and wherein the expression of the one or more target genes is activated or repressed; and/or
4) the one or more effector proteins or fragments thereof are covalently linked with one or more RNA-binding polypeptides.

9.-16. (canceled)

17. The method of claim 1, further comprising:
1) selecting one or more gene sequences encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or
2) selecting one or more gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or
3) selecting one or more gene sequences encoding a third RNA molecule, wherein the third RNA molecule comprises one or more RNA aptamers and wherein the third RNA molecule binds to the one or more RNA-binding polypeptides covalently linked to two or more RNA-binding polypeptides.

18. The method of claim 17, wherein in a cell:
1) the targeted therapeutic expresses 1) one or more effector proteins or fragments thereof covalently linked to one or more RNA-binding polypeptides; 2) one or more RNA-guided molecule covalently linked to one or more RNA-binding polypeptides and one or more guide RNA (gRNA); and 3) one or more RNA molecules,
wherein the one or more RNA molecules binds to a) one or more RNA-guided molecule and one or more effector proteins or fragments thereof; b) one or more effector proteins or fragments thereof; and/or c) one or more RNA-guided molecules; and/or
2) the one or more RNA-guided molecule is guided to the target nucleic acid in the genome by the one or more guide RNA (gRNA); and/or
3) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; and/or
4) the one or more effector proteins or fragments thereof regulate the target nucleic acid.

19. (canceled)

20. The method of claim 1, wherein the one or more effector proteins or fragments thereof:
1) are selected from the group consisting of: transcription factors, DNA modification enzymes and cofactors, and histone modifying enzymes and cofactors, and epigenetic regulators; and/or
2) an entire protein, a fusion protein, an effector domain of the effector protein, or covalently linked effector domains from one or more effector proteins or fragments thereof, or a combination thereof.

21.-22. (canceled)

23. The method of claim 1, wherein the targeted therapeutic is inducible, and/or wherein the targeted therapeutic is present in a composition comprising a pharmaceutical composition.

24.-28. (canceled)

29. A targeted therapeutic comprising:
a) one or more gene sequences encoding one or more effector proteins, or fragments thereof; and
b) a gene sequence encoding one or more RNA-guided molecules and a gene sequence encoding one or more guide RNAs (gRNA), wherein the RNA-guided molecule comprises a dead Cas9 (dCas9) protein, and wherein the gRNA comprises either 1) a crispr RNA (crRNA) and a trans-activating crispr RNA (tracrRNA), or 2) a single guide RNA (sgRNA), wherein the RNA-guided molecule and gRNA interact with a target nucleic acid in the genome, and wherein the one or more effector proteins or fragments thereof are guided by the RNA-guided molecule/gRNA complex to the target nucleic acid in the genome, wherein the targeted therapeutic comprises one or more nucleic acid constructs.

30.-35. (canceled)

36. The targeted therapeutic of claim 29,
1) wherein the gRNA further comprises one or more RNA aptamers, wherein the one or more RNA aptamers are capable of binding one or more RNA-binding polypeptides; and/or
2) the RNA-guided molecule further comprises one or more RNA-binding polypeptides, wherein the RNA-guided molecule is covalently linked to the one or more RNA-binding polypeptides; and/or
3) the target nucleic acid in the genome is non-coding DNA at or adjacent to one or more target genes, wherein the non-coding DNA includes cis-regulatory elements (CREs), and wherein the expression of the one or more target genes is activated or repressed; and/or
4) the one or more effector proteins or fragments thereof are covalently linked with one or more RNA-binding polypeptides.

37.-44. (canceled)

45. The targeted therapeutic of claim 29, further comprising:
1) one or more gene sequences encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or
2) one or more gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or
3) one or more gene sequences encoding a third RNA molecule, wherein the third RNA molecule comprises one or more RNA aptamers and wherein the third RNA molecule binds to the one or more RNA-binding polypeptides covalently linked to two or more RNA-binding polypeptides.

46. The targeted therapeutic of claim 45, wherein in a cell:
1) the targeted therapeutic expresses 1) one or more effector proteins or fragments thereof covalently linked to one or more RNA-binding polypeptides; 2) one or more RNA-guided molecules covalently linked to one or more RNA-binding polypeptides and one or more guide RNAs (gRNA); and 3) one or more RNA molecules,
wherein the one or more RNA molecules binds to a) one or more RNA-guided molecules and one or more effector proteins or fragments thereof; b) one or more effector proteins or fragments thereof; and/or c) one or more RNA-guided molecules; and/or
2) the one or more RNA-guided molecules is guided to the target nucleic acid in the genome by the one or more guide RNAs (gRNA);
3) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule;
4) the one or more effector proteins or fragments thereof regulate the target nucleic acid.

47. (canceled)

48. The targeted therapeutic of claim 29,
1) wherein the one or more effector proteins or fragments thereof are selected from the group consisting of: transcription factors, DNA modification enzymes and cofactors, and histone modifying enzymes and cofactors, and epigenetic regulators; and/or
2) wherein one or more effector or fragments thereof proteins are an entire protein, a fusion protein, an effector domain of the effector protein, or covalently linked effector domains from one or more effector proteins or fragments thereof, or a combination thereof; and/or
3) wherein the targeted therapeutic is inducible, and/or
4) wherein the targeted therapeutic is present in a composition comprising a pharmaceutical composition.

49.-58. (canceled)

59. A method of regulating gene transcription in a cell, a method comprising:
contacting the cell with a targeted therapeutic, wherein the targeted therapeutic comprises i) a gene sequences encoding one or more effector proteins or fragments thereof; and ii) a gene sequence encoding one or more RNA-guided molecules comprising a dead Cas9 (dCas9) protein and a gene sequence encoding one or more guide RNAs (gRNA); and wherein the RNA-guided molecule and gRNA interact with a target nucleic acid in the genome, and wherein the one or more effector proteins or fragments thereof are guided by the RNA-guided molecule/gRNA complex to the target nucleic acid in the genome.

60.-65. (canceled)

66. The method of claim 59,
1) wherein the gRNA further comprises one or more RNA aptamers, wherein the one or more RNA aptamers are capable of binding one or more RNA-binding polypeptides; and/or
2) the RNA-guided molecule further comprises one or more RNA-binding polypeptides, wherein the RNA-guided molecule is covalently linked to the one or more RNA-binding polypeptides; and/or
3) the target nucleic acid in the genome is non-coding DNA at or adjacent to one or more target genes, wherein the non-coding DNA includes cis-regulatory elements (CREs), and wherein the expression of the one or more target genes is activated or repressed; and/or
4) the one or more effector proteins or fragments thereof are covalently linked with one or more RNA-binding polypeptides.

67.-74. (canceled)

75. The method of claim 59, wherein the targeted therapeutic further comprises
1) a gene sequence encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or
2) a gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or
3) a gene sequence encoding a third RNA molecule, wherein the third RNA molecule comprises one or more RNA aptamers and wherein the third RNA molecule binds to the one or more RNA-binding polypeptides covalently linked to two or more RNA-binding polypeptides.

76. The method of claim 75,
1) the targeted therapeutic expresses 1) one or more effector proteins or fragments thereof covalently linked to one or more RNA-binding polypeptides; 2) one or more RNA-guided molecule covalently linked to one or more RNA-binding polypeptides and one or more guide RNA (gRNA); and 3) one or more RNA molecules,
wherein the one or more RNA molecules binds to a) one or more RNA-guided molecule and one or more effector proteins or fragments thereof; b) one or more effector proteins or fragments thereof; and/or c) one or more RNA-guided molecules; and/or
2) the one or more RNA-guided molecule is guided to the target nucleic acid in the genome by the one or more guide RNA (gRNA); and/or
3) the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; and/or
4) the one or more effector proteins or fragments thereof regulate the target nucleic acid.

77. (canceled)

78. The method of claim 59,
1) wherein the one or more effector proteins or fragments thereof are selected from the group consisting of: transcription factors, DNA modification enzymes and cofactors, and histone modifying enzymes and cofactors, and epigenetic regulators; and/or
2) wherein one or more effector or fragments thereof proteins are an entire protein, a fusion protein, an effector domain of the effector protein, or covalently linked effector domains from one or more effector proteins or fragments thereof, or a combination thereof; and/or
3) wherein the targeted therapeutic is inducible, and/or
4) wherein the targeted therapeutic is present in a composition comprising a pharmaceutical composition.

79.-89. (canceled)

90. A method of treating disease in a subject, a method comprising:
administering to the subject a targeted therapeutic, wherein the targeted therapeutic comprises i) a gene sequences encoding one or more effector proteins or fragments thereof; and ii) a gene sequence encoding one or more RNA-guided molecules and a gene sequence encoding one or more guide RNAs (gRNA), wherein the RNA-guided molecule comprises a dead Cas9 (dCas9) protein; and wherein the RNA-guided molecule and gRNA interact with a target nucleic acid in the genome, and wherein the one or more effector proteins or fragments thereof are guided by the RNA-guided molecule/gRNA complex to the target nucleic acid in the genome; and
wherein the target nucleic acid is associated with disease.

91.-96. (canceled)

97. The method of claim 90,
1) wherein the gRNA further comprises one or more RNA aptamers, wherein the one or more RNA aptamers are capable of binding one or more RNA-binding polypeptides; and/or
2) the RNA-guided molecule further comprises one or more RNA-binding polypeptides, wherein the RNA-guided molecule is covalently linked to the one or more RNA-binding polypeptides; and/or
3) the target nucleic acid in the genome is non-coding DNA at or adjacent to one or more target genes, wherein the non-coding DNA includes cis-regulatory elements (CREs), and wherein the expression of the one or more target genes is activated or repressed; and/or
4) the one or more effector proteins or fragments thereof are covalently linked with one or more RNA-binding polypeptides.

98.-105. (canceled)

106. The method of claim 90, wherein the targeted therapeutic further comprises
1) a gene sequence encoding a first RNA molecule, wherein the first RNA molecule comprises one or more RNA aptamers and wherein the first RNA molecule binds to the one or more RNA-binding polypeptides covalently linked to the one or more RNA-guided molecules and one or more RNA-binding polypeptides covalently linked to the one or more effector proteins or fragments thereof; and/or
2) a gene sequences encoding a second RNA molecule, wherein the second RNA molecule comprises one or more RNA aptamers and wherein the second RNA molecule binds to the one or more RNA-binding polypeptides covalently linked to the two or more effector proteins; and/or
3) a gene sequence encoding a third RNA molecule, wherein the third RNA molecule comprises one or more RNA aptamers and wherein the third RNA molecule binds to the one or more RNA-binding polypeptides covalently linked to two or more RNA-binding polypeptides; and/or
wherein the targeted therapeutic expresses 1) one or more effector proteins or fragments thereof covalently linked to one or more RNA-binding polypeptides; 2) one or more RNA-guided molecules covalently linked to one or more RNA-binding polypeptides and one or more guide RNAs (gRNA); and 3) one or more RNA molecules,
wherein the one or more RNA molecules binds to a) one or more RNA-guided molecules and one or more effector proteins or fragments thereof; b) one or more effector proteins or fragments thereof; and/or c) one or more RNA-guided molecules; and
wherein the one or more RNA-guided molecules is guided to the target nucleic acid in the genome by the one or more guide RNAs (gRNA); the one or more RNA molecules binds to the RNA-binding polypeptide covalently linked to the RNA-guided molecule and i) the RNA-binding polypeptide covalently linked to one or more effector proteins or fragments thereof; and/or ii) a RNA-binding polypeptide covalently linked to a second RNA-guided molecule; and the one or more effector proteins or fragments thereof regulate the target nucleic acid.

107.-109. (canceled)

110. The method of claim 90,
1) wherein the one or more effector proteins or fragments thereof are selected from the group consisting of: transcription factors, DNA modification enzymes and cofactors, and histone modifying enzymes and cofactors, and epigenetic regulators; and/or
2) wherein one or more effector or fragments thereof proteins are an entire protein, a fusion protein, an effector domain of the effector protein, or covalently linked effector domains from one or more effector proteins or fragments thereof, or a combination thereof; and/or
3) wherein the targeted therapeutic is inducible, and/or
4) wherein the targeted therapeutic is present in a composition comprising a pharmaceutical composition.

111.-121. (canceled)


SEQUENCE LISTING (12 sequences)

SEQ ID NO: 1 - DNA, 2121 bp, synthetic construct
  misc_feature 1..2121: note = Description of Artificial Sequence: Synthetic polynucleotide
  source 1..2121: mol_type = other DNA
atggcagatacacctaccttgttcacacagtttctgcggcaccatttgcctggccagcggttcaggaaggacatcctgaagcaagcagggcgcatactggctaacaagggcgaagacgccactatagctttccttagaggcaagagcgaggagagtcctcccgactttcagccacccgtgaagtgtccaattattgcatgttccagaccccttacagagtggcccatttaccaggcgtccgtggcaatccaaggttatgtttacgggcagtccctggccgagttcgaggccagtgaccccggctgcagcaaggatggcctgttaggatggtttgacaagaccggtgtgtgcaccgattatttttctgtgcaagggttaaatcttatttttcagaatgcccgcaagcgctatatcggggtgcagactaaagtaacaaacagaaacgaaaaacgccacaagaaactgaagcggatcaacgcaaaacggatcgccgaaggtctgcccgagcttactagtgacgagccagagagcgcccttgatgagacgggtcacctgatagatccacccggactgaacacgaatatatattgctatcagcaggtctcccccaagcctctggccttgagtgaggttaatcagttgcctacagcttacgcgggttattctacctcaggggacgaccctattcaaccgatggtaactaaagaccgtctgtccatctcaaaggggcagccgggatacattcctgaacaccagagggccctgctatcacagaaaaaacaccgccgaatgcggggctatgggctcaaagctagagctctgctcgttatagtgcggatccaggatgactgggccgtcattgatctgcgctctctgctcaggaacgcgtactggcgaagaattgtgcagaccaaagagccgtccacaatcaccaaactgctgaagctggtgaccggggaccctgtcctggatgccaccaggatggtagctacatttacatataagcccggaatcgtccaggtgcggtcagccaagtgtttaaaaaataagcaaggcagcaagttgttttcggaacggtacctgaacgagaccgtaagtgtgacctctattgcactgggaagtaacaatttggttgccgtggcaacctacagattggtgaatggaaatactcccgaactcctgcagcgcttcactttgccgtcacacttagtcaaagatttcgagcgctacaagcaggcacacgacactctcgaggatagcatccagaagacagctgtggcgtctctcccccaaggccaacaaactgagatacgcatgtggagcatgtacggcttcagggaagcccaggagcgagtgtgccaggaactaggtctggctgacggctccatcccatggaacgtgatgacggctacttctactattttgactgatctctttctagcaagggggggcgacccaaagaaatgcatgtttaccagcgagccaaaaaaaaagaaaaacagcaagcaggtcttatataaaattagagatagagcctgggctaaaatgtacaggacccttctcagcaaggaaacgcgcgaagcatggaataaggctctctggggacttaaacgaggatcgccagattacgccaggcttagcaagcgaaaggaggagctcgcccggagatgtgttaattacacaatctccacagccgagaaaagagcccagtgtggaaggaccattgttgctctagaagacctcaatatcggcttctttcatggacgggggaagcaggagccaggatgggtcggactattcacacgtaagaaagagaacaggtggctgatgcaagccctccataaggccttcctggaacttgcacatcatcgagggtatcacgtcatcgaagtcaatcccgcgtacacctctcagacgtgtcctgtgtgcagacactgcgacccagataatagggatcaacacaaccgtgaagcattccattgcatcggctgtggcttccggggtaacgccgacctggacgtagctacccataacattgctatggtggccatcaccggcgaatcactcaagagagcacgtggctccgttgcttctaaaacacctcagcctctggcggccgaa

SEQ ID NO: 2 - DNA, 2271 bp, synthetic construct
  misc_feature 1..2271: note = Description of Artificial Sequence: Synthetic polynucleotide
  source 1..2271: mol_type = other DNA
atgccaaaacccgccgtggagtctgaatttagtaaggttttgaagaagcattttcccggtgaacgcttccggagcagctatatgaagcgcgggggaaaaatcttagccgctcagggggaagaagccgtagtggcataccttcagggtaagtctgaggaggagcccccaaactttcagcctcccgcgaagtgccacgtggtgaccaaatctagagattttgctgaatggccgatcatgaaggctagcgaggccattcaaaggtatatttacgcactctccactaccgagagagccgcatgtaaacctggcaaatcatccgagagtcacgctgcatggttcgctgcaaccggcgttagcaatcacggatacagccatgttcagggactgaacctgatatttgaccatacactgggacggtacgacggcgttttgaagaaggtgcagctgcgcaatgagaaagcacgagccaggctggagtctatcaacgcctcccgggccgacgaaggccttccggaaattaaggccgaggaagaggaagtggccaccaatgaaacaggacatctgctccagccacccggtattaaccctagtttttatgtgtaccagacaatctcaccccaggcctatcggccgagggacgaaatcgtgctgccacctgaatatgcgggatatgtaagagatcctaacgcaccaataccacttggagtggtgcggaataggtgcgatatccagaagggctgtcccggctacataccggagtggcaacgcgaagcgggcaccgcaatcagcccaaagacgggaaaggccgtcacggtgccagggcttagccctaaaaagaataaaagaatgaggcggtattggaggagtgagaaagagaaggctcaggacgcacttttggtcacagtccggattggtacagactgggtggtcatcgatgtacgtggattgctgcgtaatgccagatggagaacaatagcgccaaaggatatctccttgaatgctctcttggatttatttacgggggatccagttattgatgttagaagaaacatagtgacctttacctatactcttgatgcctgcgggacttacgccaggaagtggacactgaagggcaaacagacaaaagcaactctcgacaagctgaccgctacgcaaaccgtcgctttagtggctattgccctgggccagaccaaccccattagtgccggaatcagcagagtcacacaagaaaacggcgcgctacaatgcgagcctctcgaccgcttcactctgcctgatgacttactgaaagacatttccgcttaccgaatcgcctgggaccgcaatgaagaagagctccgagcccgatctgtggaggccctgccagaggcacagcaagcagaagtccgagcgctggacggtgtgtccaaggagacagcgaggacccagctctgtgccgactttgggctcgatcccaaacgtctgccctgggataagatgtcatccaacacaaccttcatttccgaagccctgctcagcaatagtgtcagcagagaccaggtgttcttcactcccgctccgaagaaaggtgcgaaaaagaaggccccagtggaggtcatgcgcaaggaccggacgtgggcacgcgcctacaaacctagactgtctgtcgaagcacagaagctgaaaaatgaggcactgtgggctttaaagaggacctcgccagaatacctgaaactatcacggcgtaaagaggaattgtgtcggaggtccatcaactacgtgatcgagaagacaaggagacggacgcagtgtcagatcgtaatacccgtgattgaggacctaaacgtgcggttctttcacggatccgggaaaaggctacccgggtgggacaatttcttcaccgctaagaaggagaatcgatggtttatccaaggcctgcacaaggccttcagtgacctcagaacccaccggtctttctatgtattcgaagttcgccctgagcgcacttcgattacttgccccaagtgcggccactgcgaggttgggaaccgcgacggcgaggccttccagtgtctctcatgtggtaaaacttgcaacgctgatctcgatgtcgctacccataaccttacacaggtagcactgaccggcaaaaccatgcctaaacgcgaggaaccccgagatgcccaggggactgctcccgcacgtaaaactaagaaggctagcaaatctaaagcccctcctgccgagagggaggatcaaacacctgctcaggagccatcacagacttca

SEQ ID NO: 3 - DNA, 2298 bp, synthetic construct
  misc_feature 1..2298: note = Description of Artificial Sequence: Synthetic polynucleotide
  source 1..2298: mol_type = other DNA
atggaaaaagagattaccgaactgactaagatacgtcgcgagttcccaaataagaagttttcgtcaaccgacatgaagaaagccggcaagttactcaaggcagagggccctgacgctgtgcgtgacttcctaaactcatgccaggagattatcggcgattttaagcctcccgtgaaaaccaatatagtgagtataagccgcccctttgaggaatggccagttagcatggttggccgcgccatccaggaatactacttttctttgaccaaagaggagctggagagtgtgcatccaggcacatcctctgaggaccataagagctttttcaacattacgggacttagcaactataactacacctccgtgcaaggtctaaacctgatttttaaaaacgctaaagcgatttatgacggaacgctcgtcaaggcaaataacaaaaataagaaattagaaaagaaatttaacgagatcaatcataagagaagcctggagggccttccaataatcacacctgacttcgaagagccatttgacgagaacgggcacctaaacaacccacccggtattaaccgaaatatctatggataccaaggatgcgcagccaaggttttcgtgcctagcaaacacaagatggtctctttgcccaaagaatacgaagggtataaccgcgatcccaacctgagcctggccggttttcgcaataggttagagatccccgagggggagcccggccacgtcccttggttccagcggatggatattcccgaaggacaaatcggacacgtgaacaaaattcagcgatttaatttcgtccacggaaagaactcgggcaaggttaagttctccgacaagactggtcgggtgaagcgataccatcactccaaatataaggatgccaccaagccctacaaattcctggaggagtccaaaaaagtcagcgccctggacagcatcctggcgatcatcacaattggggacgactgggtagttttcgatatccgagggttgtataggaatgtgttctacagagagcttgctcagaagggcttgactgccgtgcaactgctggatctgttcacaggggatcctgtaattgaccccaaaaaaggagtagttaccttctcatacaaggaaggggtcgttccggtcttttcccagaagattgtgccgcgcttcaaatcccgggacaccctggaaaaacttaccagccaggggcctgtggccctgctttccgtcgctctcggccaaaatgaaccggtggccgcacgcgtgtgttcattaaagaacatcaatgataagattactctggataactcctgcaggatttccttcctcgatgactacaaaaaacagattaaggactatcgagacagcctcgatgagctggagatcaagatacggttggaagccattaattccctagagactaaccagcaggtggaaatcagggacctggatgtgttttcagccgatagggcaaaggctaatactgtggatatgtttgacatagacccaaaccttattagttgggatagcatgtctgatgcacgagtgagcacccagatctccgatctctacctgaagaatggcggggacgagagtagagtgtatttcgagatcaataacaagagaattaagcggagtgattacaacatttcccagctcgtgcggcctaaactctctgatagcacaagaaagaatctcaacgactctatatggaaactcaaacggactagtgaggagtatctgaaactgtcgaagcgcaaattagaactcagcagagctgtggtgaattatactatcaggcagtcaaagcttttgtcaggaatcaatgacatcgtaataattctcgaagacctggacgttaagaagaagttcaacggcagaggcatcagggatatagggtgggataattttttctcttctagaaaggaaaaccggtggtttattcccgcgtttcataaggcgttttctgaattgtcatctaacagggggctttgcgtgatcgaggtaaatccagcttggacaagtgccacctgtccagactgtggtttctgtagtaaagaaaatagagatggtatcaatttcacatgccgtaaatgtggagtctcttaccacgcagacatcgacgtcgccacactgaacatcgctagagtcgccgtgctgggtaagccgatgagtggacctgcagatcgggagaggctgggcgatacgaagaaacctcgtgtcgccaggtcccgcaaaaccatgaaacggaaggacatctcaaattctacggttgaagctatggtaacagct

SEQ ID NO: 4 - DNA, 2952 bp, synthetic construct
  misc_feature 1..2952: note = Description of Artificial Sequence: Synthetic polynucleotide
  source 1..2952: mol_type = other DNA
atggcacgaattcttgctttcgctatcggtatcagcagtatcgggtgggctttctcagagaacgacgaactcaaagattgtggtgttcgcatatttactaaggtagagaatccgaaaacgggagagtccctcgccctgccgcgcaggctggctcggtccgcaaggaaaagactggcccgtagaaaggcgcgattgaatcacctgaaacatttgatcgccaacgaatttaaactcaactatgaagactatcagtcattcgacgagagccttgccaaagcttataagggaagtctgatctctccctatgagttgcggtttagagccctgaatgagctgctatccaagcaggattttgctcgcgtgatcctgcatatcgctaagagaagaggctacgatgatattaagaattcagatgataaggagaagggcgccatcctcaaggcaatcaaacaaaatgaagagaagctggcaaattaccaaagtgtgggtgaatatctgtacaaggaatattttcagaagttcaaagagaactctaaggagttcaccaacgtaaggaataagaaggagtcatacgagagatgcattgcacagtcttttcttaaagatgagctgaagttgatttttaaaaaacaaagagaattcggattctcattctcaaagaaattcgaggaagaagtcctgtccgtggctttttacaagagggccttgaaggattttagccatctggttgggaattgctctttttttacggacgagaaacgcgcacctaagaattcccctctggccttcatgttcgttgcactgactcgaatcatcaatctcctgaataatctcaaaaacaccgaaggcatcctgtacacaaaggacgatctcaacgccttattgaacgaggtcctcaaaaatggcacactgacttataaacagacaaagaagcttcttggtctgtctgatgattatgagttcaaaggggagaaagggacctatttcatcgaattcaaaaagtataaggaatttattaaggctcttggggaacataacctgtctcaggacgatctgaacgaaatcgctaaggacataacattaatcaaggatgagatcaaactgaagaaggcactagcaaaatacgacttgaaccagaatcaaatcgactctttaagtaaactggaatttaaggatcaccttaacatctcttttaaagcactaaaattggtgactccacttatgttagaaggtaagaaatacgacgaagcctgcaacgagctgaatctcaaagttgccatcaatgaagataagaaagactttctgcccgcgtttaacgagacttactacaaggatgaagtgactaacccagtggtgctcagggctatcaaggaataccggaaggtccttaatgcccttctgaaaaaatatggcaaggtccacaagattaatatagagctggcacgcgaggtcggcaaaaaccattctcagcgcgccaaaatagagaaggagcagaacgagaactacaaggccaaaaaggacgccgaactcgagtgcgaaaaacttggactgaagatcaattcaaagaatattctcaagctccgtctttttaaggagcagaaagagttctgtgcctactcgggggaaaaaattaaaattagcgacctacaggacgaaaagatgctggaaatcgacgctatctatccctacagtcggtccttcgacgactcctatatgaacaaagtactggtttttacaaagcaaaaccaggaaaagctaaatcagacacctttcgaggctttcgggaatgacagcgccaaatggcagaagatagaggtgttggctaagaacttacctacgaagaaacagaaaagaatactcgacaagaactataaagataaagagcagaagaacttcaaagaccgcaaccttaacgatacaaggtacattgcacggttagtactgaattacacaaaggactatctggatttcttgcccctcagcgatgacgagaataccaagttaaatgatacgcagaagggcagtaaggtccacgtggaggctaaaagtggcatgctcacttccgcgctgaggcatacctggggattctccgcgaaagaccgtaacaatcacttacaccacgcaattgatgccgtgataattgcctatgccaacaattccattgtgaaggcctttagtgactttaagaaggagcaggaaagcaacagcgctgagctgtacgccaagaagatttccgagctcgattacaaaaacaaaaggaagttctttgagccttttagcggctttcggcaaaaggtcttggacaaaattgacgagattttcgtcagcaagcccgaaagaaaaaaacccagcggggcactccacgaagagaccttccggaaggaagaggagttctaccagagttacggcgggaaagagggagtactgaaggccctagagctcggcaagatacgaaaggttaatggcaagatagttaagaacggagatatgtttcgggtggacatctttaagcacaaaaagaccaacaagttttatgctgtgccaatctacaccatggacttcgcattgaaggtgctgccaaacaaagcggtggcgcgcagcaaaaaaggagagataaaagattggattctgatggacgagaactatgagttctgtttctcgctctacaaggactcgctaatcctgattcagactaaagacatgcaggaaccagagttcgtctactacaacgcttttacctcttcaacagttagcctgatcgtgtccaaacatgataataagttcgaaaccttgtctaagaaccaaaagattctgtttaagaatgcgaatgaaaaagaagtgatcgccaaaagcattggtattcagaacctgaaggtcttcgagaagtatattgtgtccgccctcggagaagtgaccaaggccgagttccgacaaagggaagatttcaaaaaa

SEQ ID NO: 5 - DNA, 2271 bp, synthetic construct; identical to SEQ ID NO: 2
  misc_feature 1..2271: note = Description of Artificial Sequence: Synthetic polynucleotide
  source 1..2271: mol_type = other DNA

SEQ ID NO: 6 - DNA, 3246 bp, synthetic construct
  misc_feature 1..3246: note = Description of Artificial Sequence: Synthetic polynucleotide
  source 1..3246: mol_type = other DNA
atggccgctttcaaacccaaccccatcaattatattttaggcctggcaatcggtatcgcttccgtgggatgggcaatggtggaaatcgatgaagaagaaaatcctatccgcctgatcgacttaggcgttcgggtgtttgagcgggcggaggttccgaaaaccggcgattcattggcaatggcccgccgcctcgcacgatccgttcgccggctcaccagacgaagagctcatcgtcttctgcgggcacgtcgcctgcttaagcgcgaaggtgtcttacaggccgcagacttcgatgagaacggactcatcaaaagcctccctaatactccgtggcagctgagggctgcagctctggatcgaaagcttaccccactcgagtggagcgccgtgctgctacacctgattaagcatagaggatatctgtcacagcgcaaaaacgaaggggagacggctgacaaggaactcggggccctgttaaagggtgtagctaataacgcacacgccctacagaccggggacttccgcactccagccgagctggccttgaataagtttgagaaggaatccggccacataagaaatcagcggggggattacagtcacacatttagcaggaaggaccttcaagcagagttgatcctgctcttcgagaagcagaaggagttcgggaacccacacgtgtctggtggactgaaagagggcattgaaacattgctgatgacgcagagaccagctttatctggagatgccgtccagaagatgctgggccattgcacctttgagccagccgaacccaaagctgcaaagaacacctataccgccgagcggtttatctggcttactaaactgaacaacctgcgcatcctggaacagggctctgagcgccctctgacggacacagaaagggcgacactgatggacgaaccctaccgcaagagtaaactgacatatgcccaggccaggaaattgctcggtctcgaggatactgctttttttaagggcttacggtacggcaaagacaacgcggaagctagtactctcatggaaatgaaggcataccacgccatctctcgggcactagagaaagaaggattgaaagacaaaaagtcccccctcaatctatccagtgagttgcaagacgaaataggcaccgccttctctctctttaagaccgacgaggatattaccggccgactcaaggatcgagtacagcctgaaattttggaggccctgcttaaacacatttcattcgacaaattcgttcagatttcgttaaaagccctccgtcggatcgtccccctgatggaacaggggaagcgttatgatgaggcgtgtgctgagatttatggggaccattatggcaaaaaaaacacagaagagaaaatctatctgcccccaatcccagcagatgagatccgcaatcctgtggttctaagagccctgtctcaagccagaaaagtgataaatggcgtggttaggcgatacggttctccagccaggatacacatcgaaactgctcgcgaagtggggaaaagttttaaggatcgaaaagagatcgagaaaaggcaggaggaaaacagaaaggatcgggaaaaggctgcggctaaatttcgtgagtattttcccaactttgtgggggagcccaagtcaaaggatatcctgaaacttagattgtatgaacaacagcatggaaagtgcttatactccggtaaggagatcaatcttgtgaggttgaacgagaagggctacgtcgagattgatgctgcactgcccttttcaagaacatgggatgacagcttcaacaataaggttctggtgctgggcagcgagaatcagaataagggcaatcagacaccttacgaatattttaatggaaaggacaatagcagagagtggcaggaattcaaggctcgggtggaaacttcgcggttccccagaagcaagaagcagcggattctgctgcaaaagttcgatgaggacggattcaaagagtgcaacctcaatgatactaggtacgtaaacagatttctatgtcagttcgttgcggatcacatactgctcacaggaaagggcaaacgtagagtctttgcgagtaacgggcaaatcacaaatctgctacgaggattctggggactcaggaaggtgagggccgagaatgacaggcaccatgccctggacgcagttgtggtggcctgcagtacagtcgcgatgcaacagaaaattactcgcttcgtaaggtacaaggagatgaatgcatttgatggtaagaccatcgacaaggagactggtaaagtactgcatcagaagacccatttccctcagccttgggaatttttcgcacaagaggtgatgattcgggtttttggcaaaccggacgggaaacctgagttcgaggaggccgatacgccggagaaactgcgtacccttttggctgaaaagttgtctagccggccggaggctgtgcacgagtacgtgaccccactttttgtcagcagagctccaaaccggaagatgagcggcgctcacaaagacacgctgcgatccgctaaaaggttcgtaaaacataacgagaagatttccgtcaagagggtgtggctcacagaaattaagttggccgacctggagaatatggtcaattacaagaatgggcgggagattgagctgtacgaagcccttaaggcgagactggaggcttacggaggtaacgccaaacaggccttcgatcctaaagacaatcccttctataaaaaggggggacaacttgtaaaggccgtcagggtggaaaagacccaggaatccggggttctccttaacaagaagaacgcatacactatagcagacaacggagatatggtgagagtcgatgtgttctgtaaagtcgacaaaaagggcaagaaccagtactttatcgtccctatatacgcctggcaggtggccgagaatattctcccagacatcgactgcaaggggtatcggattgacgactcatatacattctgtttctcactgcataagtatgatctgatagccttccagaaagacgaaaagagcaaagtggaatttgcctactacattaactgcgatagctctaacggccgattctatctggcctggcacgacaaagggtccaaggaacagcaatttaggatctccacccagaacttggtccttattcaaaagtaccaggtgaacgagctcggaaaagaaatacgcccttgtagattaaaaaagaggcccccagtgcgc

SEQ ID NO: 7 - DNA, 26 bp, synthetic construct
  misc_feature 1..26: note = Description of Artificial Sequence: Synthetic oligonucleotide
  source 1..26: mol_type = other DNA
ggtggtggtggttgtggtggtggtgg

SEQ ID NO: 8 - DNA, 27 bp, synthetic construct
  misc_feature 1..27: note = Description of Combined DNA/RNA Molecule: Synthetic oligonucleotide
  modified_base 1: mod_base = OTHER; note = uracil
  modified_base 4..6: mod_base = OTHER; note = uracil
  modified_base 9: mod_base = OTHER; note = uracil
  modified_base 11: mod_base = OTHER; note = uracil
  modified_base 15..16: mod_base = OTHER; note = uracil
  source 1..27: mol_type = other DNA
tgctttgatgtcgattcgacaggaggc

SEQ ID NO: 9 - DNA, 50 bp, synthetic construct
  misc_feature 1..50: note = Description of Artificial Sequence: Synthetic oligonucleotide
  source 1..50: mol_type = other DNA
cgacgcaccatttgtttaatatgttttttaattccccttgtggtgcgtcg

SEQ ID NO: 10 - DNA, 59 bp, synthetic construct
  misc_feature 1..59: note = Description of Artificial Sequence: Synthetic oligonucleotide
  source 1..59: mol_type = other DNA
ggtgtgccaataaaccatatcgccgcgttagcatgtactcggttggccctaaatacgag

SEQ ID NO: 11 - DNA, 27 bp, synthetic construct
  misc_feature 1..27: note = Description of Artificial Sequence: Synthetic oligonucleotide
  source 1..27: mol_type = other DNA
gttgcatcccaaatagaggaccgcgat

SEQ ID NO: 12 - DNA, 54 bp, synthetic construct
  misc_feature 1..54: note = Description of Artificial Sequence: Synthetic oligonucleotide
  source 1..54: mol_type = other DNA
gtcaaggctgcgcgaggttggtctacagaaaccacgctagtgtaacttgccagt